(19) United States
(12) Patent Application Publication
(10) Pub. No.: US 2024/0191186 A1
(43) Pub. Date: Jun. 13, 2024

(54) METHODS AND COMPOSITIONS FOR IPSC-DERIVED MICROGLIA

(71) Applicants: FUJIFILM Cellular Dynamics, Inc., Madison, WI (US); FUJIFILM HOLDINGS AMERICA CORPORATION, Valhalla, NY (US)

(72) Inventors: Deepika RAJESH, Madison, WI (US); Kiranmayee BAKSHY, Madison, WI (US); Christie SAVIC, Madison, WI (US); Sarah BURTON, Madison, WI (US); Wayne POON, Madison, WI (US); Simon HILCOVE, Madison, WI (US)

(73) Assignees: FUJIFILM Cellular Dynamics, Inc., Madison, WI (US); FUJIFILM HOLDINGS AMERICA CORPORATION, Valhalla, NY (US)

(21) Appl. No.: 17/760,446

(22) PCT Filed: May 5, 2022
(86) PCT No.: PCT/US2022/027842

§ 371 Date: Aug. 9, 2022

Related U.S. Application Data
(60) Provisional application No. 63/184,711, filed on May 5, 2021.

(51) Int. Cl. C12N 5/079; G01N 33/50; G01N 33/68

(52) U.S. Cl. CPC C12N 5/0622; G01N 33/5058; G01N 33/6863; C12N 2506/45; G01N 2333/535; G01N 2333/5406; G01N 2333/5437; G01N 2333/57; G01N 2400/50

(57) ABSTRACT

The present disclosure provides iPSC-derived microglia comprising a protective CD33 allele. Further provided herein are assays for screening analytes and genes associated with the protective CD33 allele.

### PRIORITY CLAIM

This application claims benefit of priority to U.S. Provisional Application Ser. No. 63/184,711, filed May 5, 2021, the entire contents of which is hereby incorporated by reference.

### BACKGROUND

### 1. Field

The present invention relates generally to the field of molecular biology and medicine. More particularly, it concerns microglia cells comprising a protective CD33 allele.

### 2. Description of Related Art

Alzheimer's disease (AD) is the most prevalent neurodegenerative disease and the leading cause of dementia among the elderly. The mechanisms underlying the onset and progression of neurodegeneration and cognitive decline are incompletely understood. A major breakthrough in the understanding of AD was the identification of gene mutations associated with rare familial AD (FAD) cases. Autosomal dominant mutations in the amyloid beta (A4) precursor protein (APP) and presenilin 1 and 2 (PSEN1/2) genes greatly accelerate the rate of cognitive decline leading to early-onset dementia.

Most AD cases are late-onset forms (LOAD) which lack an obvious Mendelian inheritance pattern. LOAD has a strong genetic component and is likely caused by a combination of multiple risk alleles, each with modest and partially penetrant effects, and environmental factors (Bertram et al., 2010).

Apolipoprotein E F4 (APOE 84) has remained for a long time the only confirmed genetic risk factor for LOAD, it accounts for only 10-20% of the LOAD risk, suggesting the existence of additional risk factors (Liu et al., 2013). Recently, genome-wide association studies (GWAS) performed on extended cohorts (thousands of individuals) led to the identification of additional confirmed genetic risk factors for AD: CD33 (Bertram et al., 2008; Hollingworth et al., 2011; Naj et al., 2011), CLU, BIN1, PICALM, CR1, CD2AP, EPHA1, ABCA7, MS4A4A/MS4A6E (Harold et al., 2009; Hollingworth et al., 2011; Lambert et al., 2009; Naj et al., 2011; Seshadri et al., 2010) and TREM2 (Guerreiro et al., 2013; Jonsson et al., 2013). However, there is an unmet need for model systems to study these genetic risk factors for a better understanding of their association with neurodegeneration and development of novel therapeutics.

### SUMMARY

Certain embodiments of the present disclosure provide an isolated induced pluripotent stem cell (iPSC)-derived microglia cell line comprising a CD33 rs12459419T allele or CD33 rs12459419C allele.

In some aspects, the cell line has an APOE 3/3 genotype. In other aspects, the cell line has an APOE 4/4 genotype. In particular aspects, the iPSC of the iPSC-derived microglia cell line is an iPSC episomally reprogrammed from a healthy donor. In specific aspects, the iPSC of the iPSC-derived microglia is an episomally reprogrammed from a donor with Alzheimer's disease. In some aspects, the cell line expresses CD45, CD11c, CD33, CD11b, and/or TREM2. In certain aspects, the cell line expresses PU.1, IBA-1, TREM2, CX3CR1, P2RY12, and/or TMEM119. In particular aspects, the cell line is isogenic.

A further embodiment provides a kit comprising a cell line of the present embodiments (e.g., an isolated induced pluripotent stem cell (iPSC)-derived microglia cell line comprising a CD33 rs12459419T allele or CD33 rs12459419C allele) in a suitable container.

In certain aspects, the kit comprises an iPSC-derived microglia cell line comprising a CD33 rs12459419T allele in a first container and an iPSC-derived microglia cell line comprising a CD33 rs12459419C allele in a second container. In some aspects, the cell line has an APOE 3/3 or APOE 4/4 genotype.

In additional aspects, the kit further comprises IFNγ, LPS, and/or GM-CSF each in a suitable container, such as a tube. In some aspects, the kit further comprises IL-4, IL-13, and/or dibutyl cAMP each in a suitable container, such as a tube. In further aspects, the kit further comprises reagents for detecting the level of IL-27, IL-10, CXCL10, CXCL11, CCL1, CCL17, CCL20, CCL22, and/or PD-1, each in a suitable container, such as enzyme-linked immunosorbent assay (ELISA) reagents. The kit may further include one or more ELISA plates.

Another embodiment provides a method for screening for a neurodegenerative disease comprising contacting an iPSC-derived microglia cell line comprising a CD33 rs12459419T allele with a sample.

In some aspects, the cell line has an APOE 3/3 genotype. In other aspects, the cell line has an APOE 4/4 genotype. In some aspects, the method further comprises contacting an iPSC-derived microglia cell line comprising a CD33 rs12459419C allele with said sample. In certain aspects, the iPSC-derived microglia cell line comprising the CD33 rs12459419T allele and/or the iPSC-derived microglia cell line comprising a CD33 rs12459419C allele are cell lines of the present embodiments and aspects thereof. In some aspects, the cell line has an APOE 3/3 genotype. In other aspects, the cell line has an APOE 4/4 genotype. In particular aspects, the iPSC of the iPSC-derived microglia cell line is an iPSC episomally reprogrammed from a healthy donor. In specific aspects, the iPSC of the iPSC-derived microglia is an episomally reprogrammed from a donor with Alzheimer's disease. In some aspects, the cell line expresses CD45, CD11c, CD33, CD11b, and/or TREM2. In certain aspects, the cell line expresses PU.1, IBA-1, TREM2, CX3CR1, P2RY12, and/or TMEM119. In particular aspects, the cell line is isogenic. In some aspects, the sample is a patient sample, such as a blood sample. In some aspects, the sample comprises a library of molecules, such as synthesized small molecules.

In additional aspects, the method further comprises detecting the level of IL-27, IL-10, CXCL10, CXCL11, CCL1, CCL17, CCL20, CCL22, and/or PD-1. In some aspects, decreased levels of IL-27, IL-10, CXCL10, CXCL11, CCL1, CCL17, CCL20, CCL22, and/or PD-1 indicate the presence of a neurodegenerative disease. For example, the neurodegenerative disease is Alzheimer's disease, Parkinson's disease, Huntington's disease, or multiple sclerosis.

A further embodiment provides a method for screening a test compound comprising introducing the test compound to a microglia cell line of the present embodiments (e.g., an isolated induced pluripotent stem cell (iPSC)-derived microglia cell line comprising a CD33 rs12459419T allele or CD33 rs12459419C allele) and measuring levels of analytes.

In some aspects, the method further comprises measuring amyloid beta phagocytic function. In certain aspects, the microglia cell population is further introduced to at least one pro-inflammatory (M1) agent or an anti-inflammatory (M2) agent. For example, the pro-inflammatory (M1) agent is LPS, IFNγ, and/or GM-CSF. In some aspects, the anti-inflammatory (M2) agent is IL-4, IL-13, IL-10 and/or dibutyl cAMP. In particular aspects, the analytes are selected from the group consisting of IL-27, IL-10, CXCL10, CXCL11, CCL1, CCL17, CCL20, CCL22, and PD-1. In some aspects, the analytes are IL-27, IL-10, CXCL10, CXCL11, CCL1, CCL17, CCL20, CCL22, and PD-1. In certain aspects, an agent that increases the level of IL-27, IL-10, CXCL10, CXCL11, CCL1, CCL17, CCL20, CCL22, and/or PD-1 is an anti-beta amyloid agent. In additional aspects, the method further comprises administering the anti-beta amyloid agent to a subject in an amount effective to prevent or decrease amyloid accumulation. In some aspects, the subject is APOE 4/4 positive.

In yet another embodiment, there is provided a method of identifying a subject at risk for neurodegeneration comprising determining an expression level of at least 10 genes from Table 1A and at least 10 genes Table 1B in a blood sample, wherein a subject with decreased expression of genes in Table 1A and increased expression of genes in Table 1B as compared to a control is at risk for neurodegeneration.

In certain aspects, the at least 10 genes in Table 1A are TENM4, MTND1P23, GREM1, GPAT2, AC243772.3, CD300E, FN1, SLC1A1, TNC, and NPPC. In specific aspects, the at least 10 genes in Table 1B are MMP2, MAG, FCER1A, CYTL1, PDCD1, ZNF90, HS3ST2, CST7, NT5DC4, and AQP1. In some aspects, the neurodegeneration is associated with Alzheimer's disease, Parkinson's disease, Huntington's disease, or multiple sclerosis. In particular aspects, determining the expression level comprises performing reverse transcription-quantitative real-time PCR (RT-qPCR), microarray analysis, Nanostring® nCounter assay, picodroplet targeting and reverse transcription, or RNA sequencing. In additional aspects, the method further comprises administering an effective amount of a therapy to said subject identified to be a risk for neurodegeneration. In certain aspects, the therapy is a cholinesterase inhibitor or anti-inflammatory agent.

A further embodiment provides a method for performing high-throughput screening to identify a therapeutic agent comprising contacting a cell line of the present embodiments (e.g., an isolated induced pluripotent stem cell (iPSC)-derived microglia cell line comprising a CD33 rs12459419T allele or CD33 rs12459419C allele) with a plurality of candidate agents and measuring levels of analytes.

In some aspects, the analytes are IL-27, IL-10, CXCL10, CXCL11, CCL1, CCL17, CCL20, CCL22, and/or PD-1. In certain aspects, the method further comprises measuring amyloid beta phagocytic function.

Another embodiments provides a co-culture comprising a microglia cell line of the present embodiments (e.g., an isolated induced pluripotent stem cell (iPSC)-derived microglia cell line comprising a CD33 rs12459419T allele or CD33 rs12459419C allele), and endothelial cells, pericytes, astrocytes, and/or neural precursor cells.

Further provided herein is the use of a co-culture of a microglia cell line of the present embodiments (e.g., an isolated induced pluripotent stem cell (iPSC)-derived microglia cell line comprising a CD33 rs12459419T allele or CD33 rs12459419C allele), and endothelial cells, pericytes, astrocytes, and/or neural precursor cells as a model of a neurodegenerative disease, such as Alzheimer's disease, Parkinson's disease, Huntington's disease, or multiple sclerosis. In some aspects, the model is further defined an organ-on-a-chip.

A further embodiment provides a composition comprising a microglia cell population at least 90% positive for TREM2, CD45, CD11c, CD33, CD11b, PU.1, IBA-1, TREM2, CX3CR1, P2RY12, and/or TMEM119, wherein the microglia cell population is differentiated from iPSCs comprising a CD33 rs12459419T allele or CD33 rs12459419C allele.

In some aspects, the microglia cell population is differentiated from iPSCs comprising a CD33 rs12459419T allele. In certain aspects, the microglia cell population is differentiated from iPSCs comprising a CD33 rs12459419C allele. In some aspects, the cell line has an APOE 3/3 genotype. In other aspects, the cell line has an APOE 4/4 genotype. In particular aspects, the iPSC of the iPSC-derived microglia cell line is an iPSC episomally reprogrammed from a healthy donor. In specific aspects, the iPSC of the iPSC-derived microglia is an episomally reprogrammed from a donor with Alzheimer's disease. In some aspects, the cell line expresses CD45, CD11c, CD33, CD11b, and/or TREM2. In certain aspects, the cell line expresses PU.1, IBA-1, TREM2, CX3CR1, P2RY12, and/or TMEM119. In particular aspects, the cell line is isogenic.

A further embodiment provides the use of a composition comprising a microglia cell population at least 90% positive for TREM2, CD45, CD11c, CD33, CD11b, PU.1, IBA-1, TREM2, CX3CR1, P2RY12, and/or TMEM119, wherein the microglia cell population is differentiated from iPSCs comprising a CD33 rs12459419T allele or CD33 rs12459419C allele for screening a test compound comprising introducing the test compound to a microglia cell line of the present embodiments (e.g., an isolated induced pluripotent stem cell (iPSC)-derived microglia cell line comprising a CD33 rs12459419T allele or CD33 rs12459419C allele) and measuring levels of analytes.

In some aspects, the use further comprises measuring amyloid beta phagocytic function. In certain aspects, the microglia cell population is further introduced to at least one pro-inflammatory (M1) agent or an anti-inflammatory (M2) agent. For example, the pro-inflammatory (M1) agent is LPS, IFNγ, and/or GM-CSF. In some aspects, the anti-inflammatory (M2) agent is IL-4, IL-13, IL-10 and/or dibutyl cAMP. In particular aspects, the analytes are selected from the group consisting of IL-27, IL-10, CXCL10, CXCL11, CCL1, CCL17, CCL20, CCL22, and PD-1. In some aspects, the analytes are IL-27, IL-10, CXCL10, CXCL11, CCL1, CCL17, CCL20, CCL22, and PD-1. In certain aspects, an agent that increases the level of IL-27, IL-10, CXCL10, CXCL11, CCL1, CCL17, CCL20, CCL22, and/or PD-1 is an anti-beta amyloid agent. In additional aspects, the use further comprises administering the anti-beta amyloid agent to a subject in an amount effective to prevent or decrease amyloid accumulation. In some aspects, the subject is APOE 4/4 positive.

In yet another embodiment, there is provided the use of a composition comprising a microglia cell population at least 90% positive for TREM2, CD45, CD11c, CD33, CD11b, PU.1, IBA-1, TREM2, CX3CR1, P2RY12, and/or TMEM119, wherein the microglia cell population is differentiated from iPSCs comprising a CD33 rs12459419T allele or CD33 rs12459419C allele for identifying a subject at risk for neurodegeneration comprising determining an expression level of at least 10 genes from Table 1A and at least 10 genes Table 1B in a blood sample, wherein a subject with decreased expression of genes in Table 1A and increased expression of genes in Table 1B as compared to a control is at risk for neurodegeneration.

In certain aspects, the at least 10 genes in Table 1A are TENM4, MTND1P23, GREM1, GPAT2, AC243772.3, CD300E, FN1, SLC1A1, TNC, and NPPC. In specific aspects, the at least 10 genes in Table 1B are MMP2, MAG, FCER1A, CYTL1, PDCD1, ZNF90, HS3ST2, CST7, NT5DC4, and AQP1. In some aspects, the neurodegeneration is associated with Alzheimer's disease, Parkinson's disease, Huntington's disease, or multiple sclerosis. In particular aspects, determining the expression level comprises performing reverse transcription-quantitative real-time PCR (RT-qPCR), microarray analysis, Nanostring® nCounter assay, picodroplet targeting and reverse transcription, or RNA sequencing. In additional aspects, the use further comprises administering an effective amount of a therapy to said subject identified to be a risk for neurodegeneration. In certain aspects, the therapy is a cholinesterase inhibitor or anti-inflammatory agent.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B: (FIG. 1A) Purity profile of end stage Microglia in an APOE 3/3 background in the presence and absence of the protective allele rs12459419 T. Cryopreserved microglia were thawed directed assessed for purity by cell surface and intracellular flowcytometry. The expression of cell surface expression of CD45, CD11c, CD33, CD11b, TREM2 and intracellular markers; PU.1, IBA-1, TREM2, CX3CR1, P2RY12, and TMEM119 were quantified. (FIG. 1B) Purity profile of end stage microglia in an APOE 4/4 background in the presence and absence of the protective allele rs12459419 T. Purity profile of end stage microglia in an APOE 4/4 background in the presence and absence of CD33. Cryopreserved microglia were thawed directed assessed for purity by cell surface and intracellular flow cytometry. The expression of cell surface expression of CD45, CD11c, CD33, CD11b, TREM2 and intracellular markers; PU.1, IBA-1, TREM2, CX3CR1, P2RY12, and TMEM119 were quantified.

FIGS. 2A-2B: (FIG. 2A) Phagocytosis of pHrodo-labeled Amyloid Beta in microglia post thaw in an APOE 3/3 background in the presence and absence of protective CD33 allele rs12459419 T. (FIG. 2B) Phagocytosis of pHrodo-labeled Amyloid Beta in microglia post thaw in an APOE 4/4 background in the presence and absence of protective CD33 allele rs12459419 T.

FIGS. 3A-3K: Cryopreserved iCell Microglia were plated in microglia maintenance medium and allowed to recover for 3 days prior to stimulation for 24 hours. Supernatants were assayed using the multiplex Luminex system in technical duplicates. Fold change over unstimulated control was calculated for each cell line, then lines with the protective allele rs12459419 T were compared to non-protective lines within each APOE 3/3 or APOE 4/4 cohort for stimulation with (FIG. 3A) LPS, (FIG. 3B) IFNγ, (FIG. 3C) LPS+IFNγ (FIG. 3D) IL-4 (FIG. 3E) IL-13, (FIG. 3F) IL-10, (FIG. 3G) TGFβ, (FIG. 3H) IL-4+IL-13, (FIG. 3I) IL-4+IL-13+IL-10, (FIG. 3J) IL-4+IL-13+TGFβ, and (FIG. 3K) IL-4+dBu-cAMP. Error bars=±1 SEM.

FIGS. 4A-4K: Cryopreserved iCell Microglia were plated in microglia maintenance medium and allowed to recover for 3 days prior to stimulation for 24 hours. Supernatants were assayed using the multiplex Luminex system in technical duplicates. Fold change over unstimulated control was calculated for each cell line, then lines with the protective allele rs12459419 T were compared to non-protective lines within each APOE 3/3 or APOE 4/4 cohort for stimulation with (FIG. 4A) LPS, (FIG. 4B) IFNγ, (FIG. 4C) LPS+IFNγ (FIG. 4D) IL-4 (FIG. 4E) IL-13, (FIG. 4F) IL-10, (FIG. 4G) TGFβ, (FIG. 4H) IL-4+IL-13, (FIG. 4I) IL-4+IL-13+IL-10, (FIG. 4J) IL-4+IL-13+TGFβ, and (FIG. 4K) IL-4+dBu-cAMP. Error bars=±1 SEM.

FIGS. 5A-5K: Cryopreserved iCell Microglia were plated in microglia maintenance medium and allowed to recover for 3 days prior to stimulation for 24 hours. Supernatants were assayed using the multiplex Luminex system in technical duplicates. Fold change over unstimulated control was calculated for each cell line, then lines with the protective allele rs12459419 T were compared to non-protective lines within each APOE 3/3 or APOE 4/4 cohort for stimulation with (FIG. 5A) LPS, (FIG. 5B) IFNγ, (FIG. 5C) LPS+IFNγ (FIG. 5D) IL-4 (FIG. 5E) IL-13, (FIG. 5F) IL-10, (FIG. 5G) TGFβ, (FIG. 5H) IL-4+IL-13, (FIG. 5I) IL-4+IL-13+IL-10, (FIG. 5J) IL-4+IL-13+TGFβ, and (FIG. 5K) IL-4+dBu-cAMP. Error bars=±1 SEM.

FIG. 6: Quantification of Soluble TREMs on day 3 and 7 post thaw in microglia harboring in the presence and absence of protective CD33 allele rs12459419 T with an APOE 3/3 or APOE 4/4 background. iCell Microglia were thawed and plated at the same density in maturation media in a 96-well Primaria plate. The absolute levels of sTREM2 were quantified by collecting the supernatants on days 3 and 7 DIV from separate wells (technical triplicates). Error bars=±1 SEM.

FIGS. 7A-7J: (FIG. 7A) A summary of genes up regulated in microglia harboring the non-protective CD33 with fold change >=2 in an APOE 3/3 background. (FIG. 7B) A summary of genes down regulated in iPSC derived microglial harboring the non-protective CD33 with fold change >=2 in an APOE 3/3 background. (FIG. 7C) A summary of genes up regulated in iPSC derived microglial harboring the non-protective CD33 variant with fold change >=2 in an APOE 4/4 background. (FIG. 7D) A summary of genes downregulated in iPSC derived microglia harboring the non-protective CD33 variant with fold change >=2 in an APOE 4/4 background. (FIG. 7E) Summary of the top 10 genes that are up or down regulated in microglia in the presence and absence of the protective CD33 variant an APOE3/3 background. (FIG. 7F) Summary of the top 10 genes that are up or down regulated in microglia in the presence and absence of the protective CD33 variant an APOE4/4 background. (FIG. 7G) Summary of the pathways up and downregulated in AP0E3/3 microglia harboring the protective and nonprotective CD33 variant. (FIG. 7H) Summary of the pathways up and downregulated in AP0E4/4 microglia harboring the protective and nonprotective CD33 variant. (FIG. 7I) Summary of the pathways up and downregulated in microglia expressing the nonprotective CD33 variant. (FIG. 7J) Summary of the pathways up and downregulated in microglia expressing the protective CD33 variant.

FIGS. 8A-8B: pHrodo SE Labeled Amyloid Beta Phagocytosis. Microglia were thawed and given three days for maturation prior to being plated for the phagocytosis assay. Microglia were seeded in a 384 well plate at 5,000 cells per well. pHrodo labeled Amyloid Beta (1 μM/well) and NucSpot 488 Nuclear Stain (1:10,000) was added to the plates and wells were monitored for red and green fluorescence using the IncuCyte S3 Live Cell Imager every 4 hours. Total Red Object Integrated Intensity (RCU×μm2/image) was normalized to Total Green Count per well using IncuCyte Software (v2019B). (FIG. 8A) APOE 4/4 Microglia comprising the non-protective CD33 (rs12459419) revealed decrease phagocytosis kinetics when exposed to pHrodo Amyloid Beta as compared to protective CD33 (rs12459419). (FIG. 8B) APOE 3/3 Microglia comprising the protective CD33 (rs12459419) the highest and lowest phagocytosis kinetics when exposed to pHrodo Amyloid Beta. APOE 3/3 Microglia comprising the non-protective CD33 (rs12459419) revealed similar levels of phagocytosis as compared to C1222 Protective.

FIGS. 9A-9B: Spare Respiratory Capacity of APOE 3/3 and APOE 4/4 Microglia. Microglia were thawed and rested for three days prior to seeding for an Agilent Seahorse Assay. Microglia were seeded at 20,000 cells per well in a PDL-coated 96-well plate and rested overnight. On the day of assay, medium was exchanged for Assay Medium comprising Seahorse XF DMEM, Glucose (10 mM), Sodium Pyruvate (1 mM), and L-Glutamate (2 mM). The plate was then incubated in a 37° C. incubator with ambient CO₂ for one hour. Stock compounds of Oligomycin A (10 uM), FCCP (30 uM), and Rotenon/Antimycin A (5 uM) from an Agilent Cell Mito Stress Test Kit were prepared and loaded into the appropriate ports of a XF96 Sensor Cartridge, according to Manufacturer's Instructions. Samples were analyzed on an Agilent Seahorse Analyzer with Wave Controller software package. Cell number was determined post-assay using Hoechst nuclear dye (1:1000) and captured using an ImageXpress MetaXpress High Content Imager. Data was normalized to oxygen consumption rate (OCR) per cell. Statistical significance was determined by two-tailed t-test for p<0.05. (FIG. 9A) APOE 3/3 and (FIG. 9B) APOE 4/4 microglia comprising the protective (PR) CD33 (rs12459419) SNP revealed a statically significant OCR over microglia comprising the non-protective (NP) CD33 (rs12459419) SNP.

### DESCRIPTION OF ILLUSTRATIVE EMBODIMENTS

Immune function and in particular, tissue resident macrophages play an integral role in disease pathogenesis. For example, the neuro-immune axis and microglia, the brain resident macrophage plays an essential role in neurodegenerative disease pathobiology including Alzheimer's disease, which is supported by both Genome-wide Association Studies and Omics studies. In addition, tissue resident macrophages play important roles in the pathogenesis of NASH (Kupffer cells), AMD (sub-retinal microglia), asthma and COPD (lung alveolar macrophages), and HIV. Numerous studies have also identified lipid regulatory dysfunction contributing to retinal microglia drusen formation, atherosclerotic plaque formation (peripheral macrophages), pulmonary foam cells, and brain AD neuropathology. Understanding how lipid dysfunction of tissue resident macrophages affects homeostatic function will serve as a therapeutic avenue for a multitude of chronic diseases with an inflammation etiology.

Recent Genome Wide Association Studies (GWAS) have identified several single nucleotide polymorphisms (SNPs) within genes expressed in microglia that regulate the late-onset Alzheimer's Disease (LOAD) risk. Inheritance of APOE allele of Apolipoprotein E (APOE4) or presence of R47H mutation in triggering receptor expressed on myeloid cells 2 (TREM2) increased risk of Alzheimer's Disease (AD), while the presence of rs12459419 T variant in CD33, a Siglec family transmembrane glycoprotein is protective. The deposition of amyloid beta (AD)-containing plaques is a pathological hallmark of both FAD and LOAD. ApoE4 affects the production, clearance and aggregation of AD. Analysis of CD33 isoforms identified a common isoform lacking exon 2 (D2-CD33). The proportion of CD33 expressed as D2-CD33 correlated robustly with rs3865444 genotype. rs3865444 is in the CD33 promoter region and additional sequencing CD33 from the promoter through exon 4 identified a single polymorphism that is coinherited with rs3865444, i.e., rs12459419 in exon 2. Thus, CD33 is a microglial mRNA and that rs3865444 is a proxy SNP for rs12459419 that modulates CD33 exon 2 splicing. Exon 2 encodes the CD33 IgV domain that typically mediates sialic acid binding in SIGLEC family members. Understanding the molecular and cellular activities of the protective rs12459419 T variant in CD33 in the presence of APOE3/3 or APOE4/4 as well as their functional interactions, should greatly advance understanding of AD.

Given the genetic link between APOE4 and CD33 on LOAD and a strong correlation between the copy number of the protective CD33 rs12459419T allele with the dose-dependent decrease in AD risk, human induced pluripotent stem cells (iPSC)-derived microglia were generated to understand the synergistic contribution of these variants in the development of AD. Episomally reprogrammed iPSCs from (i) healthy donors expressing APOE3/3 genotype and (ii) AD donors expressing an APOE4/4 genotype with the CD33 protective rs12459419T allele or the non-protective rs12459419C allele were expanded and successfully differentiated into microglia. Cryopreserved microglia from all donor samples expressed microglia-specific cell markers (CD45, TREM2, CD33, P2RY12, TMEME119, CX3CR1, IBA-1). Functional assessment of end stage cryopreserved microglia displayed altered kinetics of phagocytosis and differences in soluble TREM2 levels between donors harboring either the protective rs12459419T or the rs12459419C allele. Microglia were treated with pro-inflammatory (M1) or anti-inflammatory (M2) stimuli to elucidate pathways involved during distinct phases of neural inflammation and neurorepair. Microglia derived from donors harboring the protective rs12459419T allele released higher levels of immunomodulatory M2 analytes including IL-10, IL-13, IL-12, IL-27, CCL8, CCL13 and CCL6 compared to microglia harboring the non-protective rs12459419C allele in donors harboring APOE 3/3 versus APOE4/4. These findings unveil the mechanism of the cellular responses elicited by the protective rs12459419T allele in the context of APOE genotype. This panel of iPSC-derived microglia can be used to understand the interplay of genetic variants involved in AD risk and identify therapeutic targets for AD treatment. The cells produced by the present methods may be used for disease modeling, drug discovery, and regenerate medicine.

Thus, in some embodiments, the present disclosure provides methods for the production of microglia from induced pluripotent stem cells (iPSCs), such as patient-derived iPSCs (e.g., healthy subjects or subjects with a neurodegenerative disease). Generally, the method comprises differentiating iPSCs to microglia. In some aspects, the cells are cultured on a charged surface. Specifically, the differentiation method may be in the absence of extracellular matrix (ECM) proteins. These microglia derived from patient derived iPSC provide an in vitro tool to create a more accurate model to understand complex interactions between human microglia, neurons, astrocytes in a 2D or 3D organoid systems and mimic neurogenerative diseases.

Further, in certain embodiments, the present disclosure provides cell lines comprising the protective CD33 rs12459419 T allele or the non-protective rs12459419C allele. Further provided herein are kits, models, and assays for use of these cell lines for the study of neurodegeneration as well as for diagnosis and treatment of neurodegenerative diseases, such as AD. The present methods and compositions can be used to enhance understanding of the mechanism of the protective allele rs12459419 T and other known protective alleles of CD33 to prevent the onset of AD in an APOE4/4 positive donor. Indeed, the present studies further identified proteins (e.g., soluble TREM2), cytokines and chemokines that when enhanced in an APOE4/4 background that could result in decreasing, inhibiting, or reducing beta amyloid accumulation in a subject. In particular, the present studies showed an anti-inflammatory effect of IL-27 in the presence of rs12459419 T.

In addition, the present studies found certain genes to be up-regulated or down-regulated in the iPSC-derived microglia with the protective CD33 rs12459419 T allele as compared to the non-protective rs12459419C allele. Table 1 shows the genes with at least a 2-fold difference in expression. Table 2 shows the top 10 up- and down-regulated genes. These genes may be used for detecting whether a subject has a favorable prognosis. The present disclosure provides an insight to genes up- or down-regulated and protein-based biomarker combinations that are released specifically by disease associated microglia. Thus, in some embodiments, these analytes can be used to detect the early onset of neurodegenerative disease or qualify the disease status in a patient. The present panel of normal and disease associated microglia could unveil molecular mechanisms and identify therapeutic targets to prime microglia to a pro-regenerative/non-inflammatory function to prevent the onset of neurodegenerative diseases.

TABLE 1
Gene expression in iPSC-derived microglia with
the protective CD33 rs12459419 T allele as compared
to the non-protective rs12459419C allele.

| Gene (A) | log2(FC) | Gene (B) | log2(FC) |
|---|---|---|---|
| TENM4 | 7.88915 | AQP1 | −6.71112 |
| MTND1P23 | 7.11361 | NT5DC4 | −6.60226 |
| GREM1 | 6.38236 | CST7 | −6.25734 |
| GPAT2 | 6.06401 | HS3ST2 | −5.5242 |
| AC243772.3 | 5.67692 | ZNF90 | −5.40431 |
| CD300E | 5.57543 | PDCD1 | −5.30615 |
| FN1 | 5.02953 | CYTL1 | −5.25631 |
| SLC1A2 | 4.67664 | FCER1A | −5.02404 |
| TNC | 4.57931 | MAG | −5.00822 |
| NPPC | 4.53998 | MMP2 | −4.8258 |
| NTM | 4.53313 | GPA33 | −4.82262 |
| LINC02458 | 4.52643 | LILRA2 | −4.65903 |
| CCL8 | 4.50418 | TRO | −4.63268 |
| SRPX2 | 4.44322 | BIRC7 | −4.62385 |
| IFI27 | 4.31857 | RNASE1 | −4.60854 |
| CXCL5 | 4.20467 | ANKS1B | −4.59757 |
| TBX21 | 4.20164 | KIF26B | −4.57791 |
| IGFN1 | 4.07937 | CALD1 | −4.56978 |
| PCDHGA11 | 4.071 | NUPR1 | −4.49597 |
| KIF7 | 4.06851 | AC116366.3 | −4.46422 |
| TPPP2 | 4.06265 | LGALS12 | −4.37148 |
| PCDHGB4 | 4.03671 | LILRB4 | −4.13241 |
| PTGES | 4.02964 | LINC00996 | −4.101 |
| CPSF1P1 | 4.02652 | APOL4 | −4.093 |
| DSP | 4.02141 | FOLR2 | −4.08592 |
| SLC28A3 | 3.99999 | NPW | −4.05385 |
| CD226 | 3.99864 | B4GALNT3 | −4.04588 |
| SNCB | 3.9078 | GPRC5D | −4.01824 |
| NPR1 | 3.89311 | CTLA4 | −4.00593 |
| PWP2 | 3.86575 | HP | −4.00337 |
| LGI2 | 3.834 | AZU1 | −3.9752 |
| SLC39A2 | 3.77371 | COL1A2 | −3.9549 |
| HOPX | 3.77112 | MMRN2 | −3.9535 |
| S1PR3 | 3.76016 | SRPX | −3.94903 |
| STAC2 | 3.75584 | PTP4A3 | −3.88496 |
| HOXB6 | 3.66019 | MYZAP | −3.85833 |
| SERPINE1 | 3.64195 | NTRK3 | −3.826 |
| ZNF528 | 3.62433 | DLGAP5 | −3.80815 |
| CBFA2T3 | 3.59437 | KCNJ5 | −3.8028 |
| SIGLEC12 | 3.59388 | LINC01480 | −3.75155 |
| SNTB2 | 3.58109 | RETN | −3.74199 |
| SYP | 3.57083 | GPRC5D-AS1 | −3.7356 |
| IGFBP6 | 3.54623 | HLA-DQA2 | −3.71726 |
| VNN2 | 3.5364 | AC107294.2 | −3.63275 |
| APCDD1L | 3.53119 | GNLY | −3.62117 |
| AC079466.1 | 3.53107 | GNG7 | −3.60281 |
| RAP1GAP | 3.52718 | OLFML3 | −3.60215 |
| ACTA2 | 3.52012 | CX3CR1 | −3.60177 |
| ANPEP | 3.49918 | INPP4B | −3.57451 |
| MARCO | 3.47656 | FAM178B | −3.56058 |
| ATP1A2 | 3.45388 | ORM2 | −3.56006 |
| AC108010.1 | 3.40279 | SNCG | −3.53176 |
| MX2 | 3.39305 | SPC25 | −3.5217 |
| PKD1P5 | 3.36116 | EMB | −3.51172 |
| VAMP7 | 3.34337 | WWC1 | −3.50315 |
| CCL7 | 3.30818 | H2AFY2 | −3.4996 |
| CARMIL3 | 3.30764 | BCL11A | −3.47773 |
| ZNF366 | 3.30019 | CCR6 | −3.47047 |
| FA2H | 3.28478 | TSPAN18 | −3.43517 |
| RSAD2 | 3.28298 | MYBL2 | −3.43039 |
| CRHR2 | 3.26544 | KIF15 | −3.4241 |
| PLS3 | 3.25298 | DAPL1 | −3.4005 |
| MYEOV | 3.23283 | LINC01529 | −3.39989 |
| RTN4RL2 | 3.23157 | APBA2 | −3.38266 |
| AC106865.1 | 3.22446 | TROAP | −3.37863 |
| CCL1 | 3.21001 | AL590683.1 | −3.33845 |
| NCR1 | 3.16934 | AC243960.1 | −3.31196 |
| EIF3CL | 3.15177 | TOP2A | −3.30946 |
| TACSTD2 | 3.13239 | WASF3 | −3.29365 |
| STXBP5-AS1 | 3.11715 | PRR15 | −3.28622 |
| MSC-AS1 | 3.08999 | AC106788.1 | −3.27045 |
| AC024909.3 | 3.07093 | VWA1 | −3.26749 |
| ULBP2 | 3.0444 | C2orf88 | −3.2523 |
| AC135983.2 | 3.02876 | PDE3A | −3.25197 |
| AGT | 3.01993 | PNMA8A | −3.21946 |
| SLC9A7P1 | 3.01691 | SERPINF1 | −3.20138 |
| LDB2 | 3.0091 | APCDD1 | −3.19893 |
| INSIG1 | 3.00605 | AL450306.1 | −3.19086 |
| CPE | 3.00339 | KIF2C | −3.18933 |
| SCG5 | 2.96993 | FGL1 | −3.18706 |
| MMP9 | 2.95152 | PCLAF | −3.1848 |
| AQP6 | 2.94448 | NEU4 | −3.1708 |
| AC007384.1 | 2.92247 | TMEM163 | −3.16839 |
| TNS1 | 2.91367 | KLF2 | −3.13298 |
| HOXB2 | 2.90152 | LRP5 | −3.11505 |
| GABRR2 | 2.89277 | BATF | −3.11347 |
| RBPMS2 | 2.89225 | MPDZ | −3.08922 |
| FAM198B-AS1 | 2.88941 | TESPA1 | −3.08744 |
| NEFM | 2.8776 | MEGF6 | −3.08236 |
| SLC4A3 | 2.87209 | LTF | −3.07919 |
| GBP2 | 2.87028 | LRRC39 | −3.06622 |
| PTPRN | 2.86902 | APOBEC3B | −3.05722 |
| ACPP | 2.80383 | NDC80 | −3.05044 |
| FCRLA | 2.78068 | CNR2 | −3.04547 |
| CDKN2B | 2.76349 | CXCL12 | −3.04256 |
| YAP1 | 2.75942 | CHDH | −3.04243 |
| F3 | 2.75484 | PHYHD1 | −3.02982 |
| SLC5A3 | 2.74882 | MLLT3 | −3.02738 |
| CLDN14 | 2.74689 | TSKU | −3.02371 |
| ADRA2B | 2.7311 | PDE9A | −3.02051 |
| NOTCH3 | 2.72461 | POLE2 | −3.01878 |
| AL118508.1 | 2.70984 | MMP10 | −3.0174 |
| PXDN | 2.70976 | GABRA3 | −3.00427 |
| EPOP | 2.70025 | ROR2 | −2.99829 |
| CFAP58 | 2.69656 | CHCHD6 | −2.9904 |
| LTBP2 | 2.6863 | PDE6G | −2.9746 |
| CXADR | 2.68271 | SCN4B | −2.96345 |
| AC025580.2 | 2.67019 | TAC4 | −2.9478 |
| DUSP6 | 2.65775 | KIF18B | −2.9476 |
| TMEM135 | 2.64692 | STK32B | −2.94376 |
| C6orf223 | 2.6398 | AC087632.1 | −2.92958 |
| RPS2P55 | 2.63531 | CTSF | −2.92842 |
| MUC20 | 2.63506 | CCDC136 | −2.91608 |
| LAYN | 2.63212 | APBA1 | −2.91099 |
| TMEM45A | 2.62016 | CD7 | −2.90973 |
| EMP1 | 2.62014 | HLA-DRB1 | −2.89543 |
| RFX8 | 2.61367 | ALDH1A3 | −2.88712 |
| C10orf10 | 2.60139 | TNFSF13 | −2.88012 |
| AC093627.1 | 2.59801 | CLUHP3 | −2.86635 |
| AC064805.1 | 2.58596 | HLA-DPA1 | −2.85296 |
| PLXNB1 | 2.57161 | ASRGL1 | −2.85114 |
| LIF | 2.56317 | BACE2 | −2.84881 |
| MYLK | 2.56034 | ACSM3 | −2.84474 |
| BMP6 | 2.55778 | IL12RB1 | −2.83193 |
| PTGS2 | 2.55751 | SHC2 | −2.83112 |
| GSTT2B | 2.55694 | SYT6 | −2.82274 |
| PTPN13 | 2.52998 | RAB3IL1 | −2.80706 |
| AC100861.1 | 2.52714 | DTX3 | −2.80341 |
| PAQR5 | 2.52463 | KCNMA1 | −2.79582 |
| TIMD4 | 2.51869 | CDK1 | −2.79546 |
| LINC02154 | 2.51393 | PDPN | −2.79349 |
| GRIP2 | 2.51192 | AL078590.3 | −2.7887 |
| CD200R1 | 2.49728 | PCBP3 | −2.76298 |
| BPI | 2.49675 | LINC01268 | −2.76264 |
| AL512625.1 | 2.4956 | LTC4S | −2.75247 |
| PDE2A | 2.49325 | PGA4 | −2.73868 |
| RNF128 | 2.48356 | RIMKLB | −2.72643 |
| ADGRV1 | 2.48037 | CD24 | −2.71341 |
| INHBE | 2.46951 | LAMB2 | −2.70784 |
| ASPDH | 2.46549 | ADAMTSL2 | −2.7026 |
| PVT1 | 2.46166 | CD207 | −2.69595 |
| PPP1R3G | 2.45375 | PLD4 | −2.69293 |
| CDKN1A | 2.45323 | DUSP27 | −2.69187 |
| SRCAP | 2.4508 | CCDC8 | −2.68256 |
| KITLG | 2.44696 | CENPM | −2.66974 |
| C1orf115 | 2.4392 | ACSS3 | −2.6667 |
| TNFRSF10A | 2.43853 | SLC47A1 | −2.66501 |
| ZNF229 | 2.43742 | COL6A2 | −2.64562 |
| GPC4 | 2.43283 | SYT2 | −2.62838 |
| LYZ | 2.41912 | GPRC5B | −2.62818 |
| FAM131B | 2.41109 | ITM2C | −2.62438 |
| NT5E | 2.41024 | ITGA9 | −2.60758 |
| INHBA | 2.40789 | CAMSAP3 | −2.60592 |
| SLFN5 | 2.39186 | SULT1A1 | −2.59493 |
| SAMD9 | 2.39178 | ERC2 | −2.59363 |
| TNFSF15 | 2.39175 | GINS2 | −2.58957 |
| HELZ2 | 2.38801 | ACSM5 | −2.58831 |
| LINC00622 | 2.3817 | ACCS | −2.58745 |
| NUMB | 2.37729 | PRKN | −2.58319 |
| CD248 | 2.37413 | HLA-DPB1 | −2.58071 |
| ZFP3 | 2.36955 | HLA-DRA | −2.57922 |
| HSD11B1 | 2.36472 | ETNK2 | −2.56589 |
| OAS2 | 2.35883 | NCR3 | −2.56385 |
| SORBS1 | 2.35607 | MYH10 | −2.55792 |
| LINC01010 | 2.35473 | CCDC102A | −2.55597 |
| TIMP1 | 2.35299 | C1orf127 | −2.55031 |
| SPRY4 | 2.35195 | PROC | −2.54097 |
| TUBB3 | 2.34065 | CTGF | −2.54095 |
| LEPR | 2.33443 | C2 | −2.53734 |
| GP1BA | 2.32112 | ZNF608 | −2.53133 |
| RASL12 | 2.31602 | VTN | −2.52968 |
| NUS1P1 | 2.30869 | MKI67 | −2.52854 |
| OCSTAMP | 2.30754 | TEAD4 | −2.52364 |
| CICP14 | 2.27957 | SCIN | −2.5118 |
| NES | 2.26295 | CFD | −2.51008 |
| NDRG4 | 2.25775 | S100A1 | −2.50979 |
| UTRN | 2.25329 | GYPC | −2.50661 |
| LINC01547 | 2.25157 | KCNN1 | −2.50432 |
| DOC2GP | 2.2513 | DLG5 | −2.50287 |
| TDGP1 | 2.24887 | AURKB | −2.50265 |
| HMGN2P46 | 2.24775 | RTN4R | −2.50171 |
| LINC02009 | 2.24458 | ADORA3 | −2.49875 |
| ASAP2 | 2.23761 | RYR1 | −2.49506 |
| CYP1B1 | 2.23686 | GOLGA7B | −2.4784 |
| CRABP2 | 2.22919 | TIMP3 | −2.47164 |
| THBD | 2.22898 | PARP15 | −2.47116 |
| RNF207 | 2.21352 | IGF1 | −2.46664 |
| DUSP8 | 2.20896 | PCDH12 | −2.45501 |
| ZSWIM4 | 2.20801 | HSD17B14 | −2.43952 |
| TLN2 | 2.20647 | SIGLEC8 | −2.42972 |
| COL11A2 | 2.20542 | FAM212A | −2.42966 |
| SLC7A11 | 2.20486 | BIRC5 | −2.42862 |
| KCNN4 | 2.20341 | SOCS2-AS1 | −2.42745 |
| DIXDC1 | 2.2022 | TDRD9 | −2.42173 |
| U2AF1 | 2.19296 | COL18A1 | −2.41908 |
| ZDHHC1 | 2.18481 | HLA-DQA1 | −2.40661 |
| CCR2 | 2.18471 | HLA-DOA | −2.39825 |
| ZNF718 | 2.18149 | EBI3 | −2.39338 |
| OAF | 2.18002 | TMEM255A | −2.37925 |
| PARM1 | 2.17958 | LINC01954 | −2.37896 |
| CASS4 | 2.17886 | TK1 | −2.37801 |
| FKBP1C | 2.16624 | TYMS | −2.36597 |
| NAPRT | 2.16159 | ADGRG1 | −2.35844 |
| CCL5 | 2.15573 | FAM241B | −2.352 |
| TEX41 | 2.1557 | C1orf186 | −2.35135 |
| AL031058.1 | 2.15259 | RASGEF1C | −2.35068 |
| RTL5 | 2.14891 | SOX9 | −2.35036 |
| DOCK5 | 2.14859 | AC012368.1 | −2.34796 |
| LINC02081 | 2.1408 | SCUBE2 | −2.33787 |
| ZMIZ1-AS1 | 2.14035 | STK39 | −2.33509 |
| FAM50B | 2.13323 | GTSE1 | −2.33148 |
| FGL2 | 2.12534 | STMN1 | −2.32918 |
| TRIM2 | 2.12072 | ASTN2 | −2.32014 |
| TMTC1 | 2.11846 | CTSD | −2.31467 |
| LINC00607 | 2.11646 | FABP3 | −2.31122 |
| TEAD3 | 2.11197 | MCOLN3 | −2.30819 |
| BCL6 | 2.10429 | CD180 | −2.30457 |
| CRISPLD2 | 2.10415 | KCNE1 | −2.29876 |
| ABLIM2 | 2.10163 | FAAH | −2.28427 |
| SLC12A8 | 2.09739 | OTOA | −2.28017 |
| FLT1 | 2.09735 | CDKN3 | −2.26098 |
| DBN1 | 2.0969 | IL18BP | −2.25762 |
| TYW1B | 2.09399 | GPR157 | −2.244 |
| SHE | 2.09203 | CNFN | −2.23921 |
| MSC | 2.08872 | FRG1CP | −2.22768 |
| SLC2A1 | 2.08494 | PKIG | −2.22757 |
| OAS3 | 2.07176 | PHACTR1 | −2.21797 |
| CCL22 | 2.07165 | PDE6B | −2.21583 |
| MICAL2 | 2.07094 | ADGRG5 | −2.21286 |
| SLFN13 | 2.06923 | HTRA4 | −2.21 |
| FOSL2 | 2.06757 | ICA1 | −2.20785 |
| CYP27B1 | 2.06199 | SERPINI1 | −2.20394 |
| PLPPR2 | 2.05994 | GTF2IRD2 | −2.19992 |
| SHOX2 | 2.05913 | AC104113.1 | −2.1798 |
| AL161725.1 | 2.05527 | LINC01857 | −2.17644 |
| OTUD7B | 2.05392 | TNFRSF25 | −2.1698 |
| MAST4 | 2.05262 | CD74 | −2.16869 |
| EPS8L2 | 2.05075 | MCF2L | −2.16836 |
| VCL | 2.0373 | TACC2 | −2.1682 |
| DNM1 | 2.03705 | IFITM10 | −2.16359 |
| AC027117.1 | 2.03617 | AC060814.4 | −2.16358 |
| SIX5 | 2.03517 | SMIM24 | −2.15902 |
| AC126755.1 | 2.02916 | DOC2A | −2.15798 |
| MT1H | 2.02461 | RNASE6 | −2.15272 |
| MRO | 2.01737 | KLKB1 | −2.14449 |
| CDKN2A | 2.01667 | MLPH | −2.14345 |
| RFPL2 | 2.01318 | NCAPG | −2.14333 |
| FCGR3B | 2.0074 | ZCCHC12 | −2.14229 |

TABLE 2
Top 10 up- and down-regulated genes.

| Gene | AKA | |
|---|---|---|
| TENM4 | Teneurin | Involved in neural development, regulating the |
| | Transmembrane | establishment of proper connectivity within the nervous |
| | Protein 4/ | system. Plays a role in the establishment of the anterior- |
| | KIAA1302, | posterior axis during gastrulation. Regulates the |
| | ODZ4, TNM4 | differentiation and cellular process formation of |
| | | oligodendrocytes and myelination of small-diameter |
| | | axons in the central nervous system (CNS). Promotes |
| | | activation of focal adhesion kinase. |
| MTND1P23 | MT-ND1 | Unprocessed pseudogene. |
| | pseudogene 23 | |
| GREM1 | Gremlin-1/ | Cytokine that may play an important role during |
| | CKTSF1B1, | carcinogenesis and metanephric kidney organogenesis, |
| | DAND2, DRM | as a BMP antagonist required for early limb outgrowth |
| | | and patterning in maintaining the FGF4-SHH feedback |
| | | loop. Down-regulates the BMP4 signaling in a dose- |
| | | dependent manner. Acts as inhibitor of monocyte |
| | | chemotaxis. Can inhibit the growth or viability of normal |
| | | cells but not transformed cells when is overexpressed. |
| GPAT2 | Glycerol-3- | Transfers an acyl-group from acyl-ACP to the sn-1 |
| | phosphate | position of glycerol-3-phosphate producing a |
| | acyltransferase | lysophosphatidic acid (LPA), an essential step for the |
| | 2, mitochondrial | triacylglycerol (TAG) and glycerophospholipids. In vitro |
| | | also transfers an acyl-group from acyl-ACP to the LPA |
| | | producing a phosphatidic acid (PA). Prefers |
| | | arachidonoyl-CoA as the acyl donor. Required for |
| | | primary processing step during piRNA biosynthesis. |
| | | Molecular mechanisms by which it promotes piRNA |
| | | biosynthesis are unclear and do not involve its |
| | | acyltransferase activity. |
| AC243772.3 | | |
| CD300E | CMRF35-like | CD300e is a surface receptor, expressed by myeloid cells, |
| | molecule 2/ | involved in the tuning of immune responses. CD300e |
| | CD200LE, CLM2, | engagement was reported to provide the cells with |
| | CMRF35A5, | survival signals, to trigger the expression of activation |
| | IREM2 | markers and the release of pro-inflammatory cytokines. |
| | | Hence, CD300e is considered an immune activating |
| | | receptor. |
| FN1 | Fibronectin 1 | Fibronectins are involved in cell adhesion, cell motility, |
| | | opsonization, wound healing, and maintenance of cell |
| | | shape. |
| SLC1A2 | Excitatory | Sodium-dependent, high-affinity amino acid transporter |
| | amino acid | that mediates the uptake of L-glutamate and also L- |
| | transporter 2/ | aspartate and D-aspartate. Functions as a symporter that |
| | EAAT2, GLT1 | transports one amino acid molecule together with two |
| | | or three Na⁺ ions and one proton, in parallel with the |
| | | counter-transport of one K⁺ ion. |
| TNC | Tenascin C/ | Extracellular matrix protein implicated in guidance of |
| | HXB | migrating neurons as well as axons during development, |
| | | synaptic plasticity as well as neuronal regeneration, |
| | | Promotes neurite outgrowth from cortical neurons |
| | | grown on a monolayer of astrocytes. |
| NPPC | Natriuretic | Hormone which plays a role in endochondral ossification |
| | Peptide C/ | through regulation of cartilaginous growth plate |
| | CNP2/NPR-C | chondrocytes proliferation. |
| Gene | AKA | Up-regulated in non-protective |
| MMP2 | Matrix | Involved in the pathogenesis of neuroinflammatory |
| | Metallopeptidase | diseases and expansion of malignant gliomas due to |
| | 2 | facilitation of the penetration of anatomical barriers. |
| MAG | Myelin- | Cell adhesion molecule involved in myelin maintenance |
| | associated | and glia-axon interaction. |
| | glycoprotein | |
| FCER1A | High affinity | Binds to the Fc region of immunoglobulins epsilon. High |
| | immunoglobulin | affinity receptor. Responsible for initiating the allergic |
| | epsilon receptor | response. Binding of allergen to receptor-bound IgE leads |
| | subunit alpha/ | to cell activation and the release of mediators (such as |
| | FCE1A | histamine) responsible for the manifestations of allergy. |
| | | The same receptor also induces the secretion of |
| | | important lymphokines. |
| CYTL1 | Cytokine-like | |
| | protein 1/ | |
| | C4orf4 | |
| PDCD1 | Programmed cell | Inhibitory receptor on antigen activated T-cells that plays |
| | death protein 1/ | a critical role in induction and maintenance of immune |
| | PD1 | tolerance to self. |
| ZNF90 | Zinc finger | May be involved in transcriptional regulation. |
| | protein 90 | |
| HS3ST2 | Heparan Sulfate- | Sulfotransferase that utilizes 3′-phospho-5′-adenylyl |
| | Glucosamine 3- | sulfate (PAPS) to catalyze the transfer of a sulfo group to |
| | O- | an N-unsubstituted glucosamine linked to a 2-O-sulfo |
| | Sulfotransferase | iduronic acid unit on heparan sulfate. Catalyzes the O- |
| | 2/3OST2 | sulfation of glucosamine in GlcA2S-GlcNS. |
| CST7 | Cystatin-F | Inhibits papain and cathepsin L but with affinities lower |
| | | than other cystatins. May play a role in immune |
| | | regulation through inhibition of a unique target in the |
| | | hematopoietic system. |
| NT5DC4 | 5′-Nucleotidase | |
| | Domain | |
| | Containing 4 | |
| AQP1 | Aquaporin 1/ | Forms a water-specific channel that provides the plasma |
| | CHIP28 | membranes of red cells and kidney proximal tubules with |
| | | high permeability to water, thereby permitting water to |
| | | move in the direction of an osmotic gradient. |

### I. Definitions

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “essentially” is to be understood that methods or compositions include only the specified steps or materials and those that do not materially affect the basic and novel characteristics of those methods and compositions.

As used herein, a composition or media that is “substantially free” of a specified substance or material contains ≤30%, ≤20%, ≤15%, more preferably ≤10%, even more preferably ≤5%, or most preferably ≤1% of the substance or material.

The terms “substantially” or “approximately” as used herein may be applied to modify any quantitative comparison, value, measurement, or other representation that could permissibly vary without resulting in a change in the basic function to which it is related.

The term “about” means, in general, within a standard deviation of the stated value as determined using a standard analytical technique for measuring the stated value. The terms can also be used by referring to plus or minus 5% of the stated value.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

“Feeder-free” or “feeder-independent” is used herein to refer to a culture supplemented with cytokines and growth factors (e.g., TGFβ, bFGF, LIF) as a replacement for the feeder cell layer. Thus, “feeder-free” or feeder-independent culture systems and media may be used to culture and maintain pluripotent cells in an undifferentiated and proliferative state. In some cases, feeder-free cultures utilize an animal-based matrix (e.g. MATRIGEL™) or are grown on a substrate such as fibronectin, collagen, or vitronectin. These approaches allow human stem cells to remain in an essentially undifferentiated state without the need for mouse fibroblast “feeder layers.”

“Feeder layers” are defined herein as a coating layer of cells such as on the bottom of a culture dish. The feeder cells can release nutrients into the culture medium and provide a surface to which other cells, such as pluripotent stem cells, can attach.

The term “defined” or “fully-defined,” when used in relation to a medium, an extracellular matrix, or a culture condition, refers to a medium, an extracellular matrix, or a culture condition in which the chemical composition and amounts of approximately all the components are known. For example, a defined medium does not contain undefined factors such as in fetal bovine serum, bovine serum albumin or human serum albumin. Generally, a defined medium comprises a basal media (e.g., Dulbecco's Modified Eagle's Medium (DMEM), F12, or Roswell Park Memorial Institute Medium (RPMI) 1640, containing amino acids, vitamins, inorganic salts, buffers, antioxidants, and energy sources) which is supplemented with recombinant albumin, chemically defined lipids, and recombinant insulin. An example of a fully defined medium is Essential 8™ medium.

For a medium, extracellular matrix, or culture system used with human cells, the term “Xeno-Free (XF)” refers to a condition in which the materials used are not of non-human animal-origin.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

“Prophylactically treating” includes: (1) reducing or mitigating the risk of developing the disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

The term “effective,” as that term is used in the specification and/or claims, means adequate to accomplish a desired, expected, or intended result. “Effective amount,” “therapeutically effective amount” or “pharmaceutically effective amount” when used in the context of treating a patient or subject with a compound means that amount of the compound which, when administered to a subject or patient for treating or preventing a disease, is an amount sufficient to affect such treatment or prevention of the disease.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Induced pluripotent stem cells (iPSCs)” are cells generated by reprogramming a somatic cell by expressing or inducing expression of a combination of factors (herein referred to as reprogramming factors). iPSCs can be generated using fetal, postnatal, newborn, juvenile, or adult somatic cells. In certain embodiments, factors that can be used to reprogram somatic cells to pluripotent stem cells include, for example, Oct4 (sometimes referred to as Oct 3/4), Sox2, c-Myc, Klf4, Nanog, and Lin28. In some embodiments, somatic cells are reprogrammed by expressing at least two reprogramming factors, at least three reprogramming factors, or four reprogramming factors to reprogram a somatic cell to a pluripotent stem cell.

The term “extracellular matrix protein” refers to a molecule which provides structural and biochemical support to the surrounding cells. The extracellular matrix protein can be recombinant and also refers to fragments or peptides thereof. Examples include collagen and heparin sulfate.

A “three-dimensional (3-D) culture” refers to an artificially-created environment in which biological cells are permitted to grow or interact with their surroundings in all three dimensions. The 3-D culture can be grown in various cell culture containers such as bioreactors, small capsules in which cells can grow into spheroids, or non-adherent culture plates. In particular aspects, the 3-D culture is scaffold-free. In contrast, a “two-dimensional (2-D)” culture refers to a cell culture such as a monolayer on an adherent surface.

As used herein, a “disruption” of a gene refers to the elimination or reduction of expression of one or more gene products encoded by the subject gene in a cell, compared to the level of expression of the gene product in the absence of the disruption. Exemplary gene products include mRNA and protein products encoded by the gene. Disruption in some cases is transient or reversible and in other cases is permanent. Disruption in some cases is of a functional or full-length protein or mRNA, despite the fact that a truncated or non-functional product may be produced. In some embodiments herein, gene activity or function, as opposed to expression, is disrupted. Gene disruption is generally induced by artificial methods, i.e., by addition or introduction of a compound, molecule, complex, or composition, and/or by disruption of nucleic acid of or associated with the gene, such as at the DNA level. Exemplary methods for gene disruption include gene silencing, knockdown, knockout, and/or gene disruption techniques, such as gene editing. Examples include antisense technology, such as RNAi, siRNA, shRNA, and/or ribozymes, which generally result in transient reduction of expression, as well as gene editing techniques which result in targeted gene inactivation or disruption, e.g., by induction of breaks and/or homologous recombination. Examples include insertions, mutations, and deletions. The disruptions typically result in the repression and/or complete absence of expression of a normal or “wild type” product encoded by the gene. Exemplary of such gene disruptions are insertions, frameshift and missense mutations, deletions, knock-in, and knock-out of the gene or part of the gene, including deletions of the entire gene. Such disruptions can occur in the coding region, e.g., in one or more exons, resulting in the inability to produce a full-length product, functional product, or any product, such as by insertion of a stop codon. Such disruptions may also occur by disruptions in the promoter or enhancer or other region affecting activation of transcription, so as to prevent transcription of the gene. Gene disruptions include gene targeting, including targeted gene inactivation by homologous recombination.

### II. iPSC Differentiation Methods

### A. HPCs

The iPSCs can be differentiated into HPCs by methods known in the art such as described in U.S. Pat. No. 8,372,642, which is incorporated by reference herein. In one method, combinations of BMP4, VEGF, Flt3 ligand, IL-3, and GM-CSF may be used to promote hematopoietic differentiation. In certain embodiments, the sequential exposure of cell cultures to a first media to prepare iPSCs for differentiation, a second media that includes BMP4, VEGF, and FGF, followed by culture in a third media that includes Flt3 ligand, SCF, TPO, IL-3, and IL-6 can differentiate pluripotent cells into HPCs and hematopoietic cells. The second defined media can also comprise heparin. Further, inclusion of FGF-2 (50 ng/ml) in the media containing BMP4 and VEGF can enhance the efficiency of the generation of hematopoietic precursor cells from pluripotent cells.

Generally, differentiation of pluripotent cells into hematopoietic precursor cells may be performed using defined or undefined conditions. It will be appreciated that defined conditions are generally preferable in embodiments where the resulting cells are intended to be administered to a human subject. Hematopoietic stem cells may be derived from pluripotent stem cells under defined conditions (e.g., using a TeSR media), and hematopoietic cells may be generated from embryoid bodies derived from pluripotent cells. In other embodiments, pluripotent cells may be co-cultured on OP9 cells or mouse embryonic fibroblast cells and subsequently differentiated.

Pluripotent cells may be allowed to form embryoid bodies or aggregates as a part of the differentiation process. The formation of “embryoid bodies” (EBs), or clusters of growing cells, in order to induce differentiation generally involves in vitro aggregation of human pluripotent stem cells into EBs and allows for the spontaneous and random differentiation of human pluripotent stem cells into multiple tissue types that represent endoderm, ectoderm, and mesoderm origins. Three-dimensional EBs can thus be used to produce some fraction of hematopoietic cells and endothelial cells.

To promote aggregate formation, the cells may be transferred to low-attachment plates for an overnight incubation in serum-free differentiation (SFD) medium, consisting of 75% IMDM (Gibco), 25% Ham's Modified F12 (Cellgro) supplemented with 0.05% N2 and 1% B-27 without RA supplements, 200 mM 1-glutamine, 0.05 mg/ml Ascorbic Acid-2-phosphate Magnesium Salt (Asc 2-P) (WAKO), and 4.5×10⁻⁴ MTG. The next day the cells may be collected from each well and centrifuged. The cells may then be resuspended in “EB differentiation media,” which consists of SFD basal media supplemented with about 50 ng/ml bone morphogenetic factor (BMP4), about 50 ng/ml vascular endothelial growth factor (VEGF), and 50 ng/ml zb FGF for the first four days of differentiation. The cells are half fed every 48 hrs. On the fifth day of differentiation the media is replaced with a second media comprised of SFD media supplemented with 50 ng/ml stem cell factor (SCF), about 50 ng/ml Flt-3 ligand (Flt-3L), 50 ng/ml interleukin-6 (IL-6), 50 ng/ml interleukin-3 (IL-3), 50 ng/ml thrombopoieitin (TPO). The cells are half fed every 48 hrs with fresh differentiation media. The media changes are performed by spinning down the differentiation cultures at 300 g for 5 minutes and aspirating half the volume from the differentiating cultures and replenishing it with fresh media. In certain embodiments, the EB differentiation media may include about BMP4 (e.g., about 50 ng/ml), VEGF (e.g., about 50 ng/ml), and optionally FGF-2 (e.g., about 25-75 ng/ml or about 50 ng/ml). The supernatant may be aspirated and replaced with fresh differentiation medium. Alternately the cells may be half fed every two days with fresh media. The cells may be harvested at different time points during the differentiation process.

HPCs may be cultured from pluripotent stem cells using a defined medium. Methods for the differentiation of pluripotent cells into hematopoietic CD34⁺ stem cells using a defined media are described, e.g., in U.S. application Ser. No. 12/715,136 which is incorporated by reference in its entirety. It is anticipated that these methods may be used with the present disclosure.

For example, a defined medium may be used to induce hematopoietic CD34⁺ differentiation. The defined medium may contain the growth factors BMP4, VEGF, Flt3 ligand, IL-3 and/or GMCSF. Pluripotent cells may be cultured in a first defined media comprising BMP4, VEGF, and optionally FGF-2, followed by culture in a second media comprising either (Flt3 ligand, IL-3, and GMCSF) or (Flt3 ligand, IL-3, IL-6, and TPO). The first and second media may also comprise one or more of SCF, IL-6, G-CSF, EPO, FGF-2, and/or TPO. Substantially hypoxic conditions (e.g., less than 20% 02) may further promote hematopoietic or endothelial differentiation.

Cells may be substantially individualized via mechanical or enzymatic means (e.g., using a trypsin or TrypLE™). A ROCK inhibitor (e.g., H1152 or Y-27632) may also be included in the media. It is anticipated that these approaches may be automated using, e.g., robotic automation.

In certain embodiments, substantially hypoxic conditions may be used to promote differentiation of pluripotent cells into hematopoietic progenitor cells. As would be recognized by one of skill in the art, an atmospheric oxygen content of less than about 20.8% would be considered hypoxic. Human cells in culture can grow in atmospheric conditions having reduced oxygen content as compared to ambient air. This relative hypoxia may be achieved by decreasing the atmospheric oxygen exposed to the culture media. Embryonic cells typically develop in vivo under reduced oxygen conditions, generally between about 1% and about 6% atmospheric oxygen, with carbon dioxide at ambient levels. Without wishing to be bound by theory, it is anticipated that hypoxic conditions may mimic an aspect of certain embryonic developmental conditions. As shown in the below examples, hypoxic conditions can be used in certain embodiments to promote additional differentiation of induced pluripotent cells into a more differentiated cell type, such as HPCs.

The following hypoxic conditions may be used to promote differentiation of pluripotent cells into hematopoietic progenitor cells. In certain embodiments, an atmospheric oxygen content of less than about 20%, less than about 19%, less than about 18%, less than about 17%, less than about 16%, less than about 15%, less than about 14%, less than about 13%, less than about 12%, less than about 11%, less than about 10%, less than about 9%, less than about 8%, less than about 7%, less than about 6%, less than about 5%, about 5%, about 4%, about 3%, about 2%, or about 1% may be used to promote differentiation into hematopoietic precursor cells. In certain embodiments, the hypoxic atmosphere comprises about 5% oxygen gas.

Regardless of the specific medium being used in any given hematopoietic progenitor cell expansion, the medium used is preferably supplemented with at least one cytokine at a concentration from about 0.1 ng/mL to about 500 ng mL, more usually 10 ng/mL to 100 ng/mL. Suitable cytokines include but are not limited to, c-kit ligand (KL) (also called steel factor (StI), mast cell growth factor (MGF), and stem cell factor (SCF)), IL-6, G-CSF, IL-3, GM-CSF, IL-1α, IL-11 MIP-1α, LIF, c-mpl ligand/TPO, and flk2/flk3 ligand (Flt2L or Flt3L). Particularly, the culture will include at least one of SCF, Flt3L and TPO. More particularly, the culture will include SCF, Flt3L and TPO.

In one embodiment, the cytokines are contained in the media and replenished by media perfusion. Alternatively, when using a bioreactor system, the cytokines may be added separately, without media perfusion, as a concentrated solution through separate inlet ports. When cytokines are added without perfusion, they will typically be added as a 10× to 100× solution in an amount equal to one-tenth to 1/100 of the volume in the bioreactors with fresh cytokines being added approximately every 2 to 4 days. Further, fresh concentrated cytokines also can be added separately in addition, to cytokines in the perfused media.

### Exemplary HPC Differentiation Method

2D HPC differentiation: iPSCs may be maintained on MATRIGEL™ or Vitronectin in the presence of E8 and adapted to hypoxia for at least 5-10 passages. Cells are split from sub confluent iPSCs and plated at a density of 0.25 million cells/well onto Amine culture dishes in the presence Serum Free Defined (SFD) media supplemented with 5 μM blebbistatin. 24 hrs post plating SFD media supplemented with 50 ng/ml of BMP4, VEGF and FGF2 is added to the culture. The following day, fresh media is exchanged to remove blebbistatin. On the fifth day of the differentiation process, the cells are placed in media containing 50 ng/ml Flt-3 Ligand, SCF, TPO, IL3 and IL6 with 5U/ml of heparin. The cells are fed every 48 hrs throughout the differentiation process. The entire process is performed under hypoxic conditions and on charged amine plates. HPCs are quantified by the presence of CD43/CD34 cells and CFU.

3D HPC Differentiation: Cells were split from sub confluent iPSCs and plated at a density of 0.25-0.5 million cells per ml into a spinner flask in the presence of Serum Free Defined (SFD) media supplemented with 5 μM blebbistatin or 1 μM H1152. 24 hrs post plating SFD media supplemented with 50 ng/ml of BMP4, VEGF and FGF2 was exchanged. On the fifth day of the differentiation process the cells were placed in media containing 50 ng/ml Flt-3 Ligand, SCF, TPO, IL3 and IL6 with 5-10 U/ml of heparin. The cells were fed every 48 hrs throughout the differentiation process. The entire process was performed under hypoxic conditions. HPCs quantified by presence of CD43/CD34. HPCs are MACS sorted using CD34 beads.

### B. Gene Disruption

In certain aspects, TREM2, MeCP2, and/or SCNA gene expression, activity or function is disrupted in cells, such as PSCs (e.g., ESCs or iPSCs). In some embodiments, the gene disruption is carried out by effecting a disruption in the gene, such as a knock-out, insertion, missense or frameshift mutation, such as biallelic frameshift mutation, deletion of all or part of the gene, e.g., one or more exon or portion therefore, and/or knock-in. For example, the disruption can be effected be sequence-specific or targeted nucleases, including DNA-binding targeted nucleases such as zinc finger nucleases (ZFN) and transcription activator-like effector nucleases (TALENs), and RNA-guided nucleases such as a CRISPR-associated nuclease (Cas), specifically designed to be targeted to the sequence of the gene or a portion thereof.

In some embodiments, the disruption of the expression, activity, and/or function of the gene is carried out by disrupting the gene. In some aspects, the gene is disrupted so that its expression is reduced by at least at or about 20, 30, or 40%, generally at least at or about 50, 60, 70, 80, 90, or 95% as compared to the expression in the absence of the gene disruption or in the absence of the components introduced to effect the disruption.

In some embodiments, the disruption is transient or reversible, such that expression of the gene is restored at a later time. In other embodiments, the disruption is not reversible or transient, e.g., is permanent.

In some embodiments, gene disruption is carried out by induction of one or more double-stranded breaks and/or one or more single-stranded breaks in the gene, typically in a targeted manner. In some embodiments, the double-stranded or single-stranded breaks are made by a nuclease, e.g., an endonuclease, such as a gene-targeted nuclease. In some aspects, the breaks are induced in the coding region of the gene, e.g., in an exon. For example, in some embodiments, the induction occurs near the N-terminal portion of the coding region, e.g., in the first exon, in the second exon, or in a subsequent exon.

In some aspects, the double-stranded or single-stranded breaks undergo repair via a cellular repair process, such as by non-homologous end-joining (NHEJ) or homology-directed repair (HDR). In some aspects, the repair process is error-prone and results in disruption of the gene, such as a frameshift mutation, e.g., biallelic frameshift mutation, which can result in complete knockout of the gene. For example, in some aspects, the disruption comprises inducing a deletion, mutation, and/or insertion. In some embodiments, the disruption results in the presence of an early stop codon. In some aspects, the presence of an insertion, deletion, translocation, frameshift mutation, and/or a premature stop codon results in disruption of the expression, activity, and/or function of the gene.

In some embodiments, gene disruption is achieved using antisense techniques, such as by RNA interference (RNAi), short interfering RNA (siRNA), short hairpin (shRNA), and/or ribozymes are used to selectively suppress or repress expression of the gene. siRNA technology is RNAi which employs a double-stranded RNA molecule having a sequence homologous with the nucleotide sequence of mRNA which is transcribed from the gene, and a sequence complementary with the nucleotide sequence. siRNA generally is homologous/complementary with one region of mRNA which is transcribed from the gene, or may be siRNA including a plurality of RNA molecules which are homologous/complementary with different regions. In some aspects, the siRNA is comprised in a polycistronic construct. In particular aspects, the siRNA suppresses both wild-type and mutant protein translation from endogenous mRNA.

In some embodiments, the disruption is achieved using a DNA-targeting molecule, such as a DNA-binding protein or DNA-binding nucleic acid, or complex, compound, or composition, containing the same, which specifically binds to or hybridizes to the gene. In some embodiments, the DNA-targeting molecule comprises a DNA-binding domain, e.g., a zinc finger protein (ZFP) DNA-binding domain, a transcription activator-like protein (TAL) or TAL effector (TALE) DNA-binding domain, a clustered regularly interspaced short palindromic repeats (CRISPR) DNA-binding domain, or a DNA-binding domain from a meganuclease. Zinc finger, TALE, and CRISPR system binding domains can be engineered to bind to a predetermined nucleotide sequence, for example via engineering (altering one or more amino acids) of the recognition helix region of a naturally occurring zinc finger or TALE protein. Engineered DNA binding proteins (zinc fingers or TALEs) are proteins that are non-naturally occurring. Rational criteria for design include application of substitution rules and computerized algorithms for processing information in a database storing information of existing ZFP and/or TALE designs and binding data. See, for example, U.S. Pat. Nos. 6,140,081; 6,453,242; and 6,534,261; see also WO 98/53058; WO 98/53059; WO 98/53060; WO 02/016536 and WO 03/016496 and U.S. Publication No. 2011/0301073.

In some embodiments, the DNA-targeting molecule, complex, or combination contains a DNA-binding molecule and one or more additional domain, such as an effector domain to facilitate the repression or disruption of the gene. For example, in some embodiments, the gene disruption is carried out by fusion proteins that comprise DNA-binding proteins and a heterologous regulatory domain or functional fragment thereof. In some aspects, domains include, e.g., transcription factor domains such as activators, repressors, co-activators, co-repressors, silencers, oncogenes, DNA repair enzymes and their associated factors and modifiers, DNA rearrangement enzymes and their associated factors and modifiers, chromatin associated proteins and their modifiers, e.g. kinases, acetylases and deacetylases, and DNA modifying enzymes, e.g. methyltransferases, topoisomerases, helicases, ligases, kinases, phosphatases, polymerases, endonucleases, and their associated factors and modifiers. See, for example, U.S. Patent Application Publication Nos. 2005/0064474; 2006/0188987 and 2007/0218528, incorporated by reference in their entireties herein, for details regarding fusions of DNA-binding domains and nuclease cleavage domains. In some aspects, the additional domain is a nuclease domain. Thus, in some embodiments, gene disruption is facilitated by gene or genome editing, using engineered proteins, such as nucleases and nuclease-containing complexes or fusion proteins, composed of sequence-specific DNA-binding domains fused to or complexed with non-specific DNA-cleavage molecules such as nucleases.

In some aspects, these targeted chimeric nucleases or nuclease-containing complexes carry out precise genetic modifications by inducing targeted double-stranded breaks or single-stranded breaks, stimulating the cellular DNA-repair mechanisms, including error-prone nonhomologous end joining (NHEJ) and homology-directed repair (HDR). In some embodiments the nuclease is an endonuclease, such as a zinc finger nuclease (ZFN), TALE nuclease (TALEN), and RNA-guided endonuclease (RGEN), such as a CRISPR-associated (Cas) protein, or a meganuclease.

In some embodiments, a donor nucleic acid, e.g., a donor plasmid or nucleic acid encoding the genetically engineered antigen receptor, is provided and is inserted by HDR at the site of gene editing following the introduction of the DSBs. Thus, in some embodiments, the disruption of the gene and the introduction of the antigen receptor, e.g., CAR, are carried out simultaneously, whereby the gene is disrupted in part by knock-in or insertion of the CAR-encoding nucleic acid.

In some embodiments, no donor nucleic acid is provided. In some aspects, NHEJ-mediated repair following introduction of DSBs results in insertion or deletion mutations that can cause gene disruption, e.g., by creating missense mutations or frameshifts.

### 1. ZFPs and ZFNs

In some embodiments, the DNA-targeting molecule includes a DNA-binding protein such as one or more zinc finger protein (ZFP) or transcription activator-like protein (TAL), fused to an effector protein such as an endonuclease. Examples include ZFNs, TALEs, and TALENs.

In some embodiments, the DNA-targeting molecule comprises one or more zinc-finger proteins (ZFPs) or domains thereof that bind to DNA in a sequence-specific manner. A ZFP or domain thereof is a protein or domain within a larger protein that binds DNA in a sequence-specific manner through one or more zinc fingers, regions of amino acid sequence within the binding domain whose structure is stabilized through coordination of a zinc ion. The term zinc finger DNA binding protein is often abbreviated as zinc finger protein or ZFP. Among the ZFPs are artificial ZFP domains targeting specific DNA sequences, typically 9-18 nucleotides long, generated by assembly of individual fingers.

ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3 and 6) on a zinc finger recognition helix. Thus, in some embodiments, the ZFP or ZFP-containing molecule is non-naturally occurring, e.g., is engineered to bind to a target site of choice.

In some aspects, disruption of MeCP2 is carried out by contacting a first target site in the gene with a first ZFP, thereby disrupting the gene. In some embodiments, the target site in the gene is contacted with a fusion ZFP comprising six fingers and the regulatory domain, thereby inhibiting expression of the gene.

In some embodiments, the step of contacting further comprises contacting a second target site in the gene with a second ZFP. In some aspects, the first and second target sites are adjacent. In some embodiments, the first and second ZFPs are covalently linked. In some aspects, the first ZFP is a fusion protein comprising a regulatory domain or at least two regulatory domains.

In some embodiments, the first and second ZFPs are fusion proteins, each comprising a regulatory domain or each comprising at least two regulatory domains. In some embodiments, the regulatory domain is a transcriptional repressor, a transcriptional activator, an endonuclease, a methyl transferase, a histone acetyltransferase, or a histone deacetylase.

In some embodiments, the ZFP is encoded by a ZFP nucleic acid operably linked to a promoter. In some aspects, the method further comprises the step of first administering the nucleic acid to the cell in a lipid:nucleic acid complex or as naked nucleic acid. In some embodiments, the ZFP is encoded by an expression vector comprising a ZFP nucleic acid operably linked to a promoter. In some embodiments, the ZFP is encoded by a nucleic acid operably linked to an inducible promoter. In some aspects, the ZFP is encoded by a nucleic acid operably linked to a weak promoter.

In some embodiments, the target site is upstream of a transcription initiation site of the gene. In some aspects, the target site is adjacent to a transcription initiation site of the gene. In some aspects, the target site is adjacent to an RNA polymerase pause site downstream of a transcription initiation site of the gene.

In some embodiments, the DNA-targeting molecule is or comprises a zinc-finger DNA binding domain fused to a DNA cleavage domain to form a zinc-finger nuclease (ZFN). In some embodiments, fusion proteins comprise the cleavage domain (or cleavage half-domain) from at least one Type 1iS restriction enzyme and one or more zinc finger binding domains, which may or may not be engineered. In some embodiments, the cleavage domain is from the Type 1iS restriction endonuclease Fok I. Fok I generally catalyzes double-stranded cleavage of DNA, at 9 nucleotides from its recognition site on one strand and 13 nucleotides from its recognition site on the other.

In some embodiments, ZFNs target a gene present in the engineered cell. In some aspects, the ZFNs efficiently generate a double strand break (DSB), for example at a predetermined site in the coding region of the gene. Typical regions targeted include exons, regions encoding N terminal regions, first exon, second exon, and promoter or enhancer regions. In some embodiments, transient expression of the ZFNs promotes highly efficient and permanent disruption of the target gene in the engineered cells. In particular, in some embodiments, delivery of the ZFNs results in the permanent disruption of the gene with efficiencies surpassing 50%.

Many gene-specific engineered zinc fingers are available commercially. For example, Sangamo Biosciences (Richmond, CA, USA) has developed a platform (CompoZr) for zinc-finger construction in partnership with Sigma-Aldrich (St. Louis, MO, USA), allowing investigators to bypass zinc-finger construction and validation altogether, and provides specifically targeted zinc fingers for thousands of proteins (Gaj et al., Trends in Biotechnology, 2013, 31(7), 397-405). In some embodiments, commercially available zinc fingers are used or are custom designed.

### 2. TALs, TALEs and TALENs

In some embodiments, the DNA-targeting molecule comprises a naturally occurring or engineered (non-naturally occurring) transcription activator-like protein (TAL) DNA binding domain, such as in a transcription activator-like protein effector (TALE) protein, See, e.g., U.S. Patent Publication No. 2011/0301073, incorporated by reference in its entirety herein.

A TALE DNA binding domain or TALE is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. Each TALE repeat unit includes 1 or 2 DNA-binding residues making up the Repeat Variable Diresidue (RVD), typically at positions 12 and/or 13 of the repeat. The natural (canonical) code for DNA recognition of these TALEs has been determined such that an HD sequence at positions 12 and 13 leads to a binding to cytosine (C), NG binds to T, NI to A, NN binds to G or A, and NO binds to T and non-canonical (atypical) RVDs are also known. See, U.S. Patent Publication No. 2011/0301073. In some embodiments, TALEs may be targeted to any gene by design of TAL arrays with specificity to the target DNA sequence. The target sequence generally begins with a thymidine.

In some embodiments, the molecule is a DNA binding endonuclease, such as a TALE nuclease (TALEN). In some aspects the TALEN is a fusion protein comprising a DNA-binding domain derived from a TALE and a nuclease catalytic domain to cleave a nucleic acid target sequence.

In some embodiments, the TALEN recognizes and cleaves the target sequence in the gene. In some aspects, cleavage of the DNA results in double-stranded breaks. In some aspects the breaks stimulate the rate of homologous recombination or non-homologous end joining (NHEJ). Generally, NHEJ is an imperfect repair process that often results in changes to the DNA sequence at the site of the cleavage. In some aspects, repair mechanisms involve rejoining of what remains of the two DNA ends through direct re-ligation (Critchlow and Jackson, 1998) or via the so-called microhomology-mediated end joining. In some embodiments, repair via NHEJ results in small insertions or deletions and can be used to disrupt and thereby repress the gene. In some embodiments, the modification may be a substitution, deletion, or addition of at least one nucleotide. In some aspects, cells in which a cleavage-induced mutagenesis event, i.e. a mutagenesis event consecutive to an NHEJ event, has occurred can be identified and/or selected by well-known methods in the art.

In some embodiments, TALE repeats are assembled to specifically target a gene. A library of TALENs targeting 18,740 human protein-coding genes has been constructed. Custom-designed TALE arrays are commercially available through Cellectis Bioresearch (Paris, France), Transposagen Biopharmaceuticals (Lexington, KY, USA), and Life Technologies (Grand Island, NY, USA).

In some embodiments the TALENs are introduced as trans genes encoded by one or more plasmid vectors. In some aspects, the plasmid vector can contain a selection marker which provides for identification and/or selection of cells which received said vector.

### 3. RGENs (CRISPR/Cas Systems)

In some embodiments, the disruption is carried out using one or more DNA-binding nucleic acids, such as disruption via an RNA-guided endonuclease (RGEN). For example, the disruption can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions.

### C. Charged Cell Surfaces

In some embodiments, the present disclosure concerns charged surfaces for cell culture. The charged surface may be positively charged, such as an amine surface or nitrogen-containing functional groups, or negatively charged, such as a carboxyl surface or oxygen-containing functional groups. The cell surfaces may be treated to alter the surface charge of the culture vessel.

In some aspects, the surface is neutrally charged, such as a surface comprising both negatively charged and positively charged functional groups. For example, the CORNING PRIMARIA® surface features a unique mixture of oxygen-containing (negatively charged) and nitrogen-containing (positive charged) functional groups on the polystyrene surface. The surface supports the growth of cells that can exhibit poor attachment or limited differentiation potential when cultured on traditional TC surfaces. In some aspects, the surface comprises a ULA surface coating. For example, the corning ultra-low attachment surface is a covalently bound hydrogel layer that is hydrophilic and neutrally charged. Since proteins and other biomolecules passively adsorb to polystyrene surfaces through either hydrophobic or ionic interactions, this hydrogel naturally inhibits nonspecific immobilization via these forces, thus inhibiting subsequent cell attachment. This surface is very stable, noncytotoxic, biologically inert and nondegradable. Other examples that could support the generation of microglia from HPCs include: Corning CellBIND culture (U.S. Pat. No. 6,617,152) uses a higher energy microwave plasma to incorporate more oxygen onto the polystyrene surface rendering it more hydrophilic (wettable) while increasing the stability of the surface compared with traditional plasma or corona discharge treated surfaces. Corning Synthemax self-coating substrate is a unique, animal-free, synthetic Vitronectin-based peptide containing the RGD motif and flanking sequences. The synthetic peptides are covalently bound to a polymer backbone for passive coating, orienting, and presenting the peptide for optimal cell binding and signaling.

The cell culture surface may be coated with a plasma polymerized film. The source of the plasma polymerization is one or more monomers. Useful polymerizable monomers may include unsaturated organic compounds such as olefinic amines, halogenated olefins, olefinic carboxylic acids and carboxylates, olefinic nitrile compounds, oxygenated olefins and olefinic hydrocarbons. In some embodiments, the olefins may include vinylic and allylic forms. In other embodiments, cyclic compounds such as cyclohexane, cyclopentane and cyclopropane may be used.

As will be recognized by those skilled in the art, various plasma polymerization techniques may be utilized to deposit the one or more monomers onto the cell culture surfaces. Preferably, a positively charged polymerized film is deposited on the surfaces. As will be appreciated by one skilled in the art, the plasma polymerized surface may have a negative charge depending on the proteins to be used therewith. Amine is preferably used as the monomer source of the polymer. In some embodiments, the plasma polymerized monomer is made using plasma sources to generate a gas discharge that provides energy to initiate polymerization of gaseous monomers and allows a thin polymer film to deposit on a culture vessel. Cyclic compounds may be utilized which may include gas plasmas by glow discharge methods. Derivatives of these cyclic compounds, such as 1,2-diaminocyclohexane for instance, are also commonly polymerizable in gas plasmas.

Mixtures of polymerizable monomers may be used. Additionally, polymerizable monomers may be blended with other gases not generally considered as polymerizable in themselves, examples being argon, nitrogen and hydrogen.

It is contemplated that any culture vessel that is useful for adherent cultures may be used. Preferred cell culture vessel configurations contemplated by the present disclosure include multiwell plates (such as 6-well, 12-well and 24-well plates), dishes (such as petri dishes), test tubes, culture flasks, roller bottles, tube or shaker flasks, and the like.

Material for the cell culture surface may include plastic (e.g. polystyrene, acrylonitrile butadiene styrene, polycarbonate); glass; microporous filters (e.g., cellulose, nylon, glass fiber, polyester, and polycarbonate); materials for bio-reactors used in batch or continuous cell culture or in genetic engineering (e.g., bioreactors), which may include hollow fiber tubes or micro carrier beads; polytetrafluoroethylene (Teflon®), ceramics and related polymeric materials.

In particular aspects, the cell culture is free of or essentially free of any extracellular matrix proteins, such as laminin, fibronectin, vitronectin, MATRIGEL™ tenascin, entactin, thrombospondin, elastin, gelatin, collagen, fibrillin, merosin, anchorin, chondronectin, link protein, bone sialoprotein, osteocalcin, osteopontin, epinectin, hyaluronectin, undulin, epiligrin, and kalinin.

### D. Differentiation of HPCs to Microglia

Microglia are innate immune cells of the central nervous system that perform critical roles in brain development, homeostasis, and immune regulation. They are hard to acquire from human fetal and primary tissues. In certain embodiments, the present methods describe the generation, characterization and cryopreservation of human iPSC-derived microglia (iMGL) from episomally reprogrammed HPCs under defined conditions. Cryopreserved iMGL retain purity, secrete immunomodulatory cytokines and phagocytose pHrodo Red labelled bacterial BioParticles and Amyloid βeta aggregates. The ability to produce essentially limitless quantities of iMGLs holds great promise for accelerating human neuroscience research into the role of microglia in normal and diseased states.

In an exemplary method, fresh or cryopreserved HPCs are thawed and plated in Microglia Differentiation Media comprising FLT-3 ligand and IL-3. The cells may be plated at a density of 10-50 K/cm2, such as 20-35 K/cm2. The Microglia Differentiation Medium may comprise IL-34, TGFβ1, or M-CSF (MDM). The culturing may be performed on MATRIGEL™ coated plate or a charged surface such as a Primaria plate or Ultra low attachment plate or a tissue culture plate (TC) or a non-tissue culture plate (Non-TC) and may be high-throughput, such as a 96 well plate (e.g., 200 μl Microglia Differentiation Medium per well). The cells may be half fed every 48 hrs with 50 μl media per well of 2× Microglia Differentiation media (MDM) the next 23 days of differentiation. In specific aspects, the differentiation is performed in the absence of ECM proteins, such as MATRIGEL®. The cells are harvested with cold PBS on day 23 and the total viable cell number is quantified using an automated cell counter. The cells are stained for surface expression of CD11b, CD11c, CD45, CD33, TREM-2 and intracellular expression of TREM-2, IBA, CX3CR1, P2RY12 and TMEM119.

In particular aspects, the present microglia were derived from iPSCs episomally reprogrammed from subjects harboring the CD33 allele rs12459419 T in the presence of an APOE3/3 (healthy) or APOe4/4 (Alzheimer's) background as well as subjects with the CD33 allele rs12459419 T in the presence of an APOE3/3 (healthy) or APOe4/4 (Alzheimer's) background. In other aspects, the CD33 allele variant microglia may be differentiated from iPSC genetically engineered to comprise the protective CD33 allele rs12459419 T allele or the non-protective CD33 allele rs12459419 C allele. The present microglia may comprise other protective or non-protective alleles of interest in neurodegenerative diseases. The microglia may be isogenically engineered cryopreserved microglia.

### E. Differentiation Media

Cells can be cultured with the nutrients necessary to support the growth of each specific population of cells. Generally, the cells are cultured in growth media including a carbon source, a nitrogen source and a buffer to maintain pH. The medium can also contain fatty acids or lipids, amino acids (such as non-essential amino acids), vitamin(s), growth factors, cytokines, antioxidant substances, pyruvic acid, buffering agents, pH indicators, and inorganic salts. An exemplary growth medium contains a minimal essential media, such as Dulbecco's Modified Eagle's medium (DMEM) or ESSENTIAL 8™ (E8™) medium, supplemented with various nutrients, such as non-essential amino acids and vitamins, to enhance stem cell growth. Examples of minimal essential media include, but are not limited to, Minimal Essential Medium Eagle (MEM) Alpha medium, Dulbecco's modified Eagle medium (DMEM), RPMI-1640 medium, 199 medium, and F12 medium. Additionally, the minimal essential media may be supplemented with additives such as horse, calf or fetal bovine serum. Alternatively, the medium can be serum free. In other cases, the growth media may contain “knockout serum replacement,” referred to herein as a serum-free formulation optimized to grow and maintain undifferentiated cells, such as stem cell, in culture. KNOCKOUT™ serum replacement is disclosed, for example, in U.S. Patent Application No. 2002/0076747, which is incorporated herein by reference. Preferably, the PSCs are cultured in a fully-defined and feeder-free media.

In some embodiments, the medium may contain or may not contain any alternatives to serum. The alternatives to serum can include materials which appropriately contain albumin (such as lipid-rich albumin, albumin substitutes such as recombinant albumin, plant starch, dextrans and protein hydrolysates), transferrin (or other iron transporters), fatty acids, insulin, collagen precursors, trace elements, 2-mercaptoethanol, 3-thioglycerol, or equivalents thereto. The alternatives to serum can be prepared by the method disclosed in International Publication No. WO 98/30679, for example. Alternatively, any commercially available materials can be used for more convenience. The commercially available materials include KNOCKOUT™ Serum Replacement (KSR), Chemically-defined Lipid concentrated (Gibco), and GLUTAMAX™ (Gibco).

Other culturing conditions can be appropriately defined. For example, the culturing temperature can be about 30 to 40° C., for example, at least or about 31, 32, 33, 34, 35, 36, 37, 38, 39° C. but particularly not limited to them. In one embodiment, the cells are cultured at 37° C. The CO₂ concentration can be about 1 to 10%, for example, about 2 to 5%, or any range derivable therein. The oxygen tension can be at least, up to, or about 1, 2, 3, 4, 5, 6, 7, 8, 9, 10, 20%, or any range derivable therein.

### F. Cryopreservation

The cells produced by the methods disclosed herein can be cryopreserved, see for example, PCT Publication No. 2012/149484 A2, which is incorporated by reference herein, at any stage of the process, such as Stage I, Stage II, or Stage III. The cells can be cryopreserved with or without a substrate. In several embodiments, the storage temperature ranges from about −50° C. to about −60° C., about −60° C. to about −70° C., about −70° C. to about −80° C., about −80° C. to about −90° C., about −90° C. to about −100° C. and overlapping ranges thereof. In some embodiments, lower temperatures are used for the storage (e.g., maintenance) of the cryopreserved cells. In several embodiments, liquid nitrogen (or other similar liquid coolant) is used to store the cells. In further embodiments, the cells are stored for greater than about 6 hours. In additional embodiments, the cells are stored about 72 hours. In several embodiments, the cells are stored 48 hours to about one week. In yet other embodiments, the cells are stored for about 1, 2, 3, 4, 5, 6, 7, or 8 weeks. In further embodiments, the cells are stored for 1, 2, 3, 4, 5, 6, 7, 8, 9, 10, 11 or 12 months. The cells can also be stored for longer times. The cells can be cryopreserved separately or on a substrate, such as any of the substrates disclosed herein.

In some embodiments, additional cryoprotectants can be used. For example, the cells can be cryopreserved in a cryopreservation solution comprising one or more cryoprotectants, such as DM80, serum albumin, such as human or bovine serum albumin. In certain embodiments, the solution comprises about 1%, about 1.5%, about 2%, about 2.5%, about 3%, about 4%, about 5%, about 6%, about 7%, about 8%, about 9%, or about 10% DMSO. In other embodiments, the solution comprises about 1% to about 3%, about 2% to about 4%, about 3% to about 5%, about 4% to about 6%, about 5% to about 7%, about 6% to about 8%, about 7% to about 9%, or about 8%. to about 10% dimethylsulfoxide (DMSO) or albumin. In a specific embodiment, the solution comprises 2.5% DMSO. In another specific embodiment, the solution comprises 10% DMSO.

Cells may be cooled, for example, at about 1° C./minute during cryopreservation. In some embodiments, the cryopreservation temperature is about −80° C. to about −180° C., or about −125° C. to about −140° C. In some embodiments, the cells are cooled to 4° C. prior to cooling at about 1° C./minute. Cryopreserved cells can be transferred to vapor phase of liquid nitrogen prior to thawing for use. In some embodiments, for example, once the cells have reached about −80° C., they are transferred to a liquid nitrogen storage area. Cryopreservation can also be done using a controlled-rate freezer. Cryopreserved cells may be thawed, e.g., at a temperature of about 25° C. to about 40° C., and typically at a temperature of about 37° C.

### III. Methods of Use

The present disclosure provides microglia (e.g., with or without a protective CD33 allele) which can be used for several important research, development, and commercial purposes. These include, but are not limited to, transplantation or implantation of the cells in vivo; screening anti-virals, cytotoxic compounds, carcinogens, mutagens, growth/regulatory factors, pharmaceutical compounds, etc., in vitro; elucidating the mechanism of neurodegenerative diseases; studying the mechanism by which drugs and/or growth factors operate; diagnosing and monitoring neurodegenerative disease in a patient; gene therapy; and the production of biologically active products, to name but a few.

### A. Pharmaceutical Compositions

Also provided herein are pharmaceutical compositions and formulations comprising the present cells and a pharmaceutically acceptable carrier.

Cell compositions for administration to a subject in accordance with the present invention thus may be formulated in any conventional manner using one or more physiologically acceptable carriers comprising excipients and auxiliaries which facilitate processing of the compounds into preparations which can be used pharmaceutically. Proper formulation is dependent upon the route of administration chosen.

Pharmaceutical compositions and formulations as described herein can be prepared by mixing the active ingredients (such as cells) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Patent Publication Nos. 2005/0260186 and 2006/0104968. In one aspect, a sHASEGP is combined with one or more additional glycosaminoglycanases such as chondroitinases.

### A. Distribution for Commercial, Therapeutic, and Research Purposes

In some embodiments, a reagent system is provided that includes cells that exists at any time during manufacture, distribution or use. The kits may comprise any combination of the cells described in the present disclosure in combination with undifferentiated pluripotent stem cells or other differentiated cell types, often sharing the same genome. Each cell type may be packaged together, or in separate containers in the same facility, or at different locations, at the same or different times, under control of the same entity or different entities sharing a business relationship. Pharmaceutical compositions may optionally be packaged in a suitable container with written instructions for a desired purpose, such as the mechanistic toxicology.

In some embodiments, a kit that can include, for example, one or more media and components for the production of cells is provided. The reagent system may be packaged either in aqueous media or in lyophilized form, where appropriate. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The components of the kit may be provided as dried powder(s). When reagents and/or components are provided as a dry powder, the powder can be reconstituted by the addition of a suitable solvent. It is envisioned that the solvent may also be provided in another container means. The kits of the present disclosure also will typically include a means for containing the kit component(s) in close confinement for commercial sale. Such containers may include injection or blow molded plastic containers into which the desired vials are retained. The kit can also include instructions for use, such as in printed or electronic format, such as digital format.

### IV. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Generation and Characterization of End Stage Microglia

CD33 encodes a member of a superfamily called sialic acid-binding immunoglobulin-like lectins (Siglecs). In microglia, CD33 binds extracellular sialylated glycans on other cells or pathogens. Its cytoplasmic domain signals via phosphatidyl-inositol-3 kinase (PI3K) to dampen microglial phagocytosis, by comparison.

The protective CD33 allele rs12459419 T was reported to alter splicing of the CD33 mRNA such that the resulting protein lacks CD33's sialic acid binding domain and therefore preserves the cell's ability to take up and clear AD. Hence, the present studies compare the phagocytic function in microglia harboring the CD33 allele rs12459419 T in the presence of an APOE3/3 (healthy) or APOe4/4 (Alzheimer's) background.

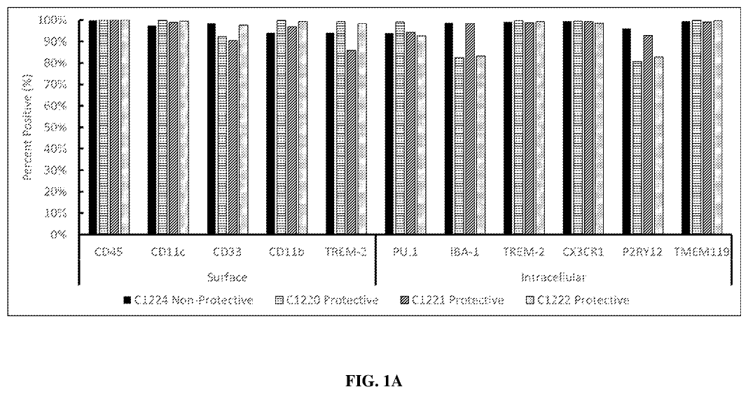

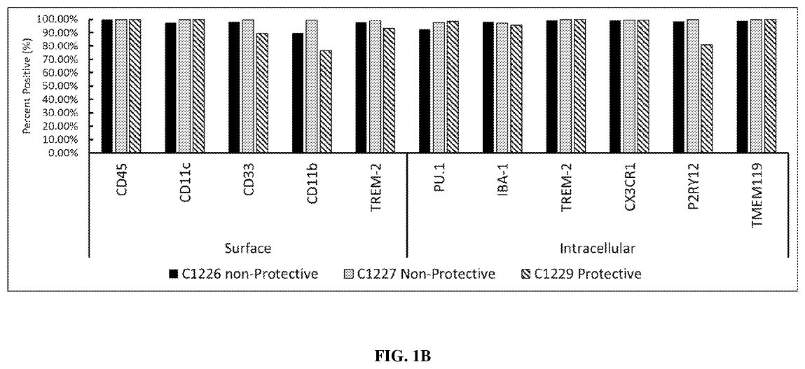

Microglia were generated from iPSC derived from healthy subjects and donors with Alzheimer's disease with an APOE3/3 vs APOE4/4 background and with and without the protective allele rs12459419 T. The cells were developed to understand the mechanism of the protective allele rs12459419 T that may be used to prevent the onset of AD in an APOE 4/4 positive donor. Cryopreserved microglia from all donor samples expressed microglia-specific cell markers (CD45, TREM2, CD33, P2RY12, TMEME119, CX3CR1, IBA-1) (FIG. 1)

TABLE 3
Apparently Heathy Normal (ANH) APOE 3/3 background
with and without protective rs12459419.

| Cell | Catalog | | | | CD33 |
|---|---|---|---|---|---|
| Line | Number | Phenotype | Genotype | APOE | (rs12459419) |
| 12024 | C1224 | AHN | NA | 3/3 | Non-protective |
| 12023 | C1220 | AHN | NA | 3/3 | Protective |
| 12056 | C1221 | AHN | NA | 3/3 | Protective |
| 12068 | C1222 | AHN | NA | 3/3 | Protective |

TABLE 4
iPSCs from Alzheimer's patients with an APOE 4/4
background with and without protective rs12459419.

| Cell | Catalog | | | | CD33 |
|---|---|---|---|---|---|
| Line | Number | Phenotype | Genotype | APOE | (rs12459419) |
| 11993 | C1226 | AD | APOE 4/4 | 4/4 | Non-protective |
| 11995 | C1227 | AD | APOE 4/4 | 4/4 | Non-protective |
| 12061 | C1229 | AD | APOE 4/4 | 4/4 | Protective |

Generation and characterization of end stage microglia from healthy subjects and donors with Alzheimer's disease with and without the protective CD33 allele rs12459419 T: All iPSCs were maintained using E8 and Matrigel and acclimatized to hypoxia for 10 passages. The iPSCs were scaled up and first differentiated into purified hematopoietic progenitor cells (HPCs). HPCs were further differentiated into end stage microglia according to the protocol described by Ebud et al. and cryopreserved. The purity of microglia was quantified post thaw by flow cytometry. In vitro differentiation of microglia was not affected by the APOE or CD33 status.

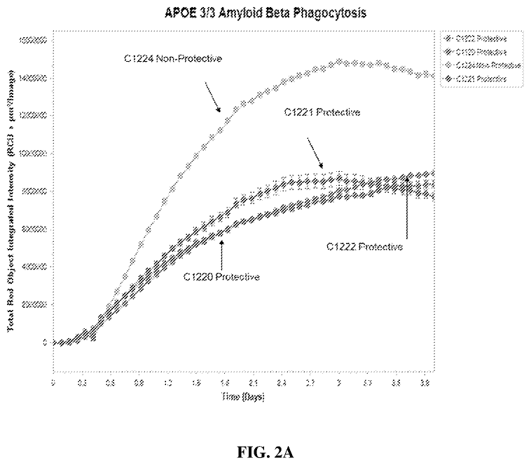

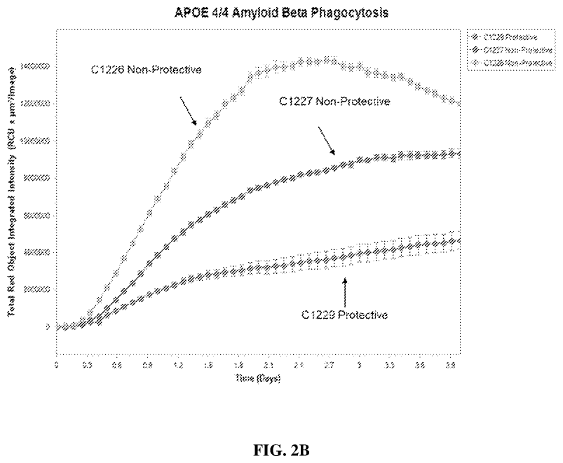

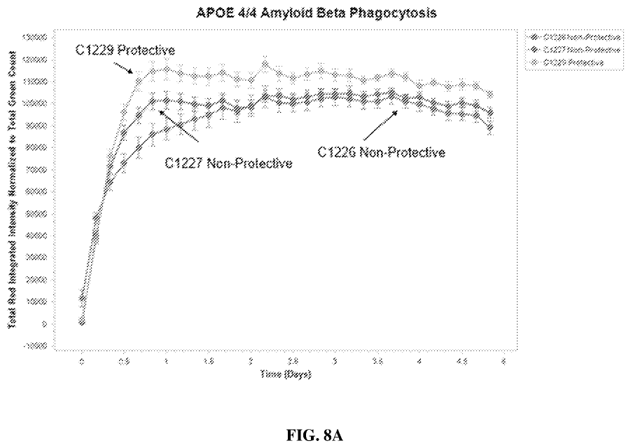

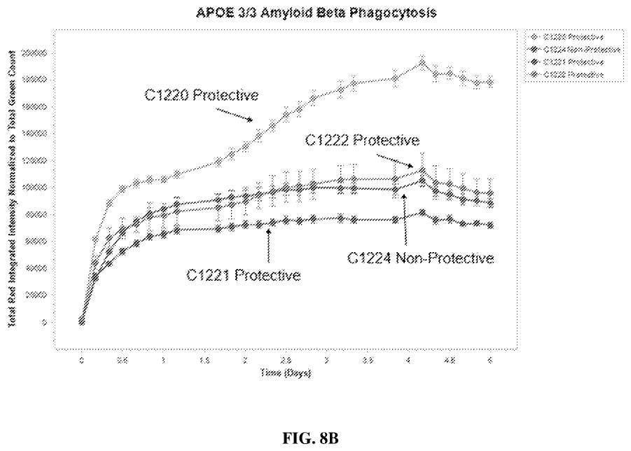

It was further found that microglia bearing the protective CD33 allele rs12459419 T had decreased phagocytosis kinetics of amyloid beta as quantified by Total Red Object Integrated Intensity (FIG. 2). When normalized to cell number it was found that microglia bearing the protective CD33 allele rs12459419 displayed altered phagocytosis kinetics (FIG. 8). Normalizing to cell number allows for the phenotypic response of the APOE 3/3 and APOE 4/4 microglia to be detected. Microglia were thawed and given three days for maturation prior to being plated for the phagocytosis assay. Microglia were seeded in a 384 well plate at 5,000 cells per well. pHrodo labeled Amyloid Beta (1 μM/well) was added to the plates and wells were monitored for red fluorescence Total Red Object Integrated Intensity (RCU×μm2/image) on an IncuCyte S3 Live Cell Imager every 2 hours. The RCU was measured and analyzed using IncuCyte Software (v2019B). The RCU was also normalized to total cell number as monitored by green fluorescence and analyzed using IncuCyte Software (v2019B). Microglia, both APOE 3/3 and APOE 4/4, containing the protective CD33 (rs12459419T) revealed increased phagocytosis kinetics when exposed to pHrodo Amyloid Beta as compared to the protective CD33 (rs12459419C), when normalized to total cell number. Thus, the protective CD33 allele rs12459419T may protect against Alzheimer's disease by maintaining a steady state of mobility and phagocytosis to amyloid beta.

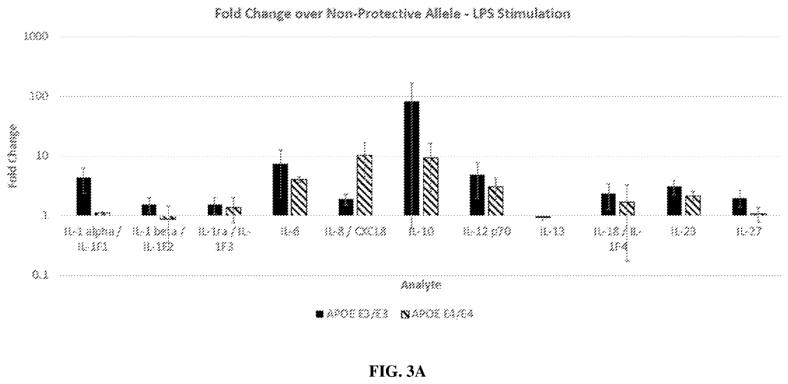

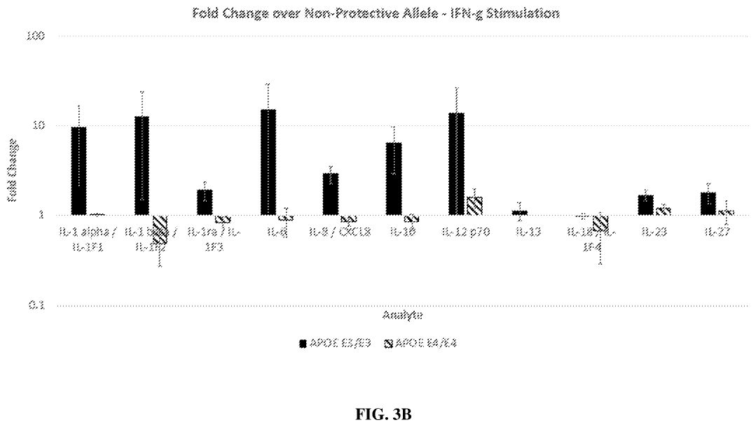

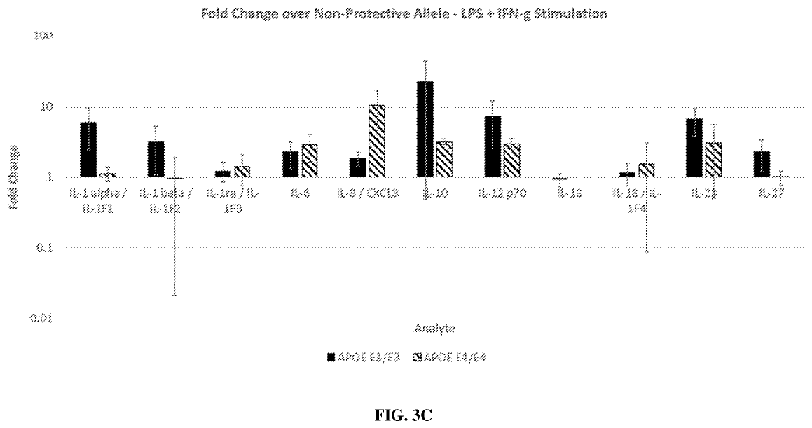

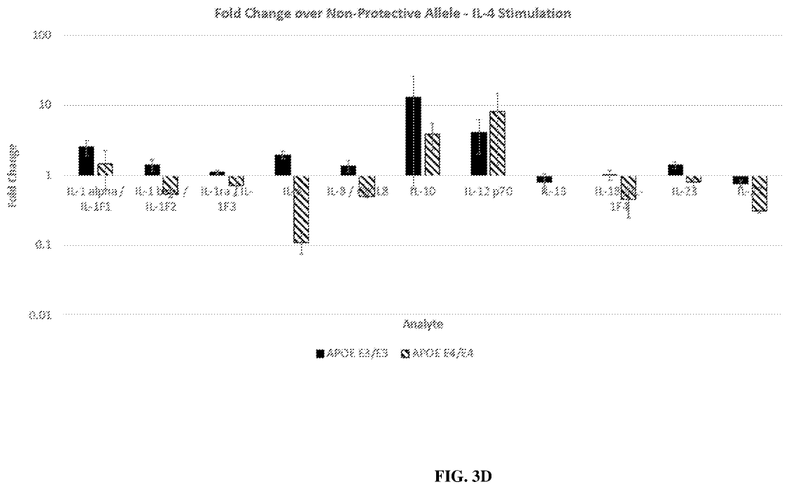

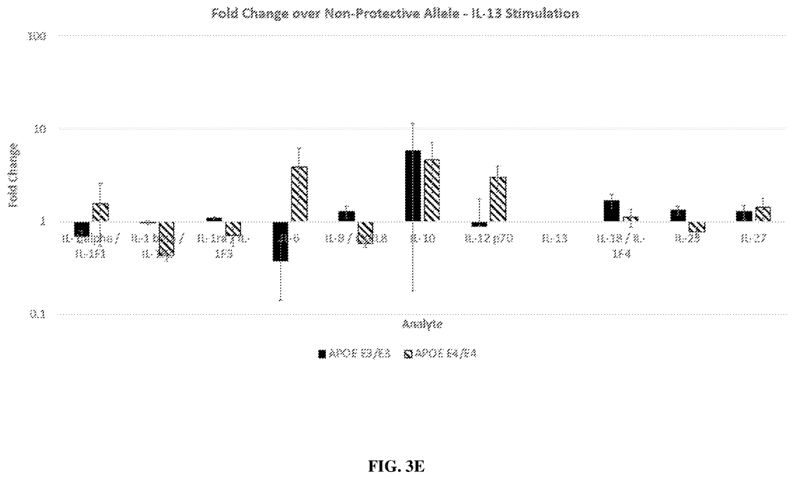

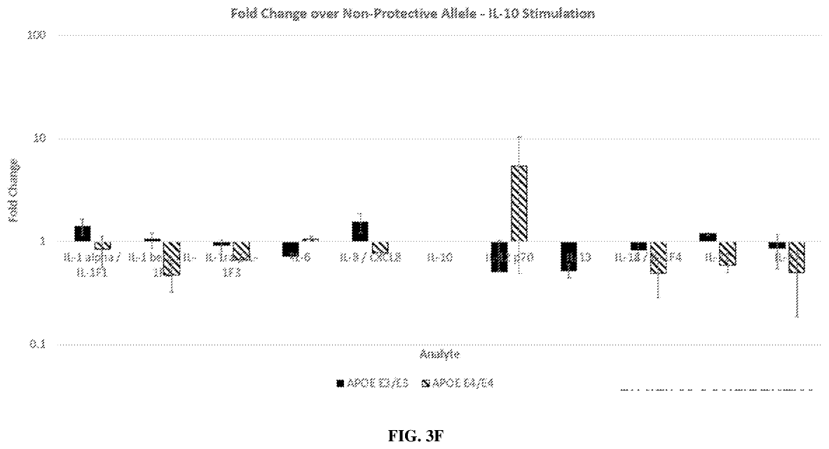

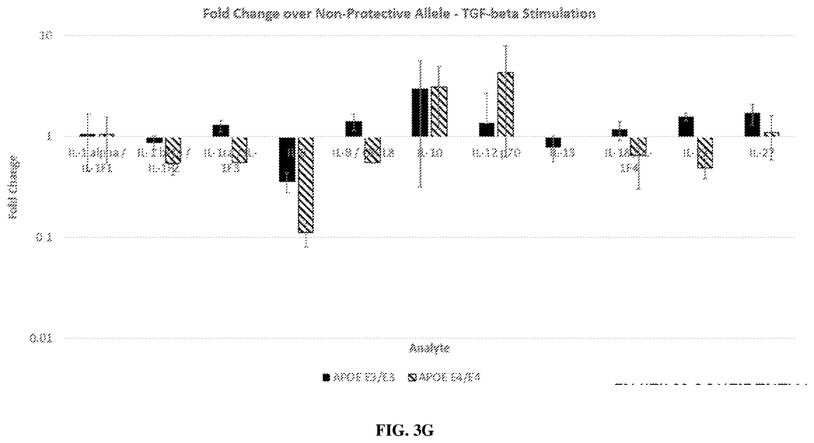

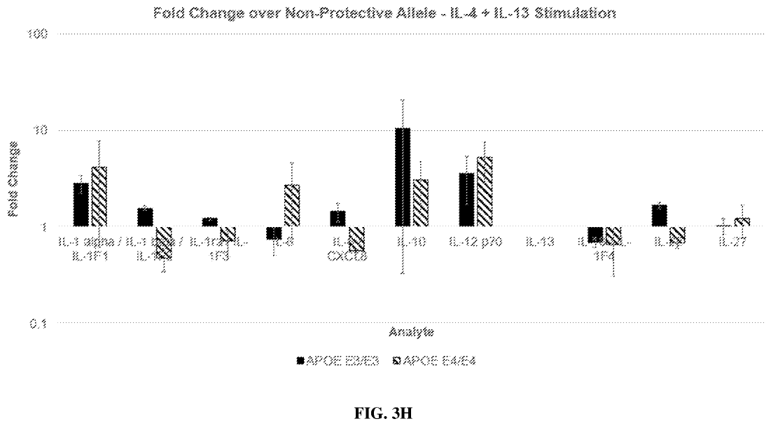

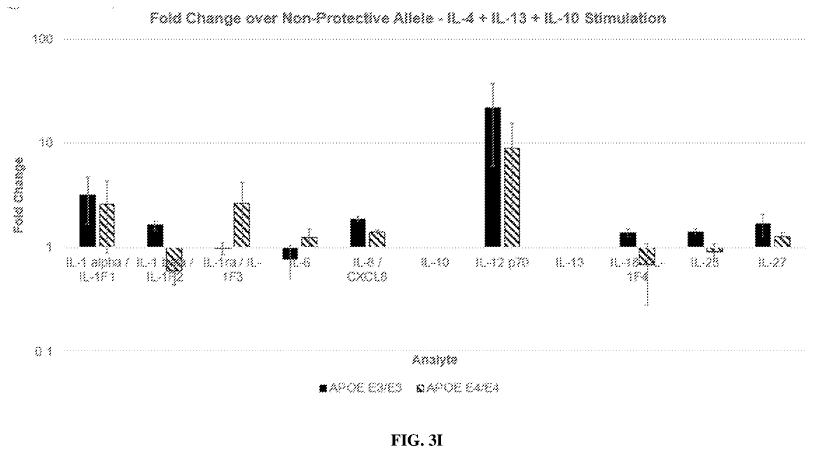

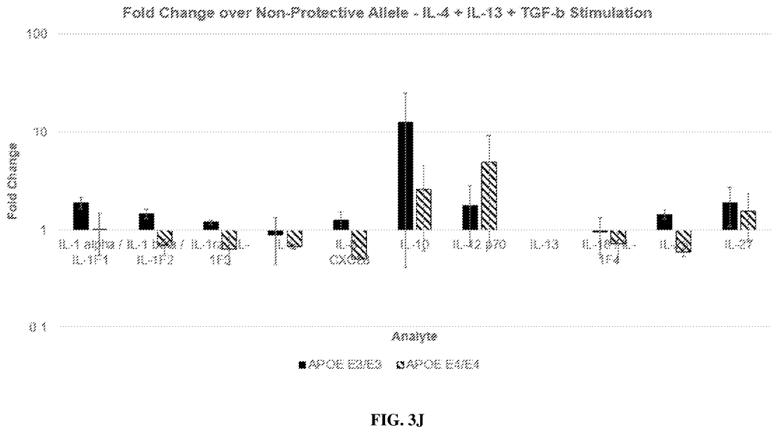

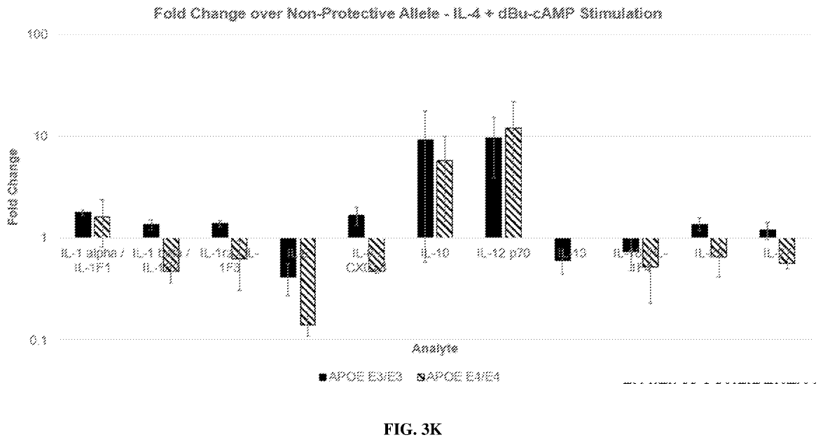

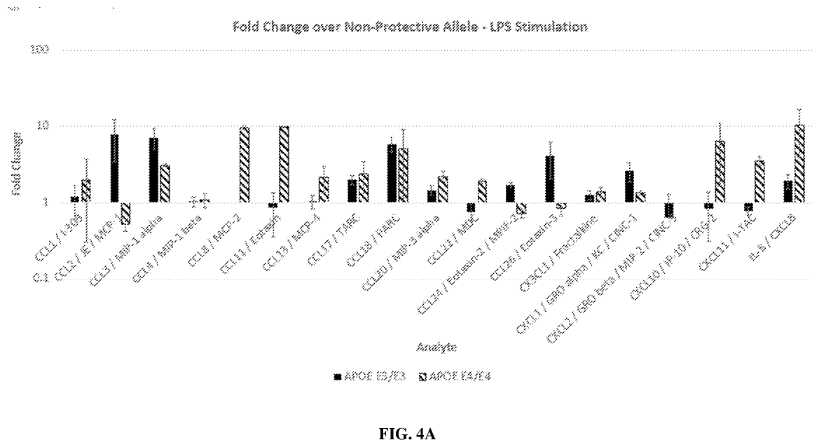

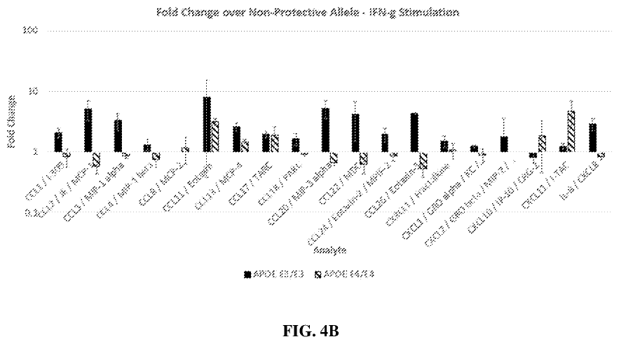

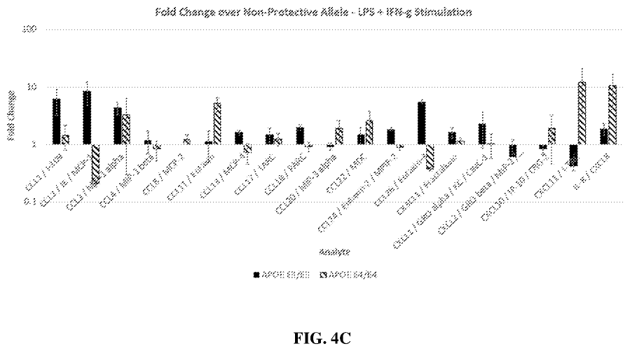

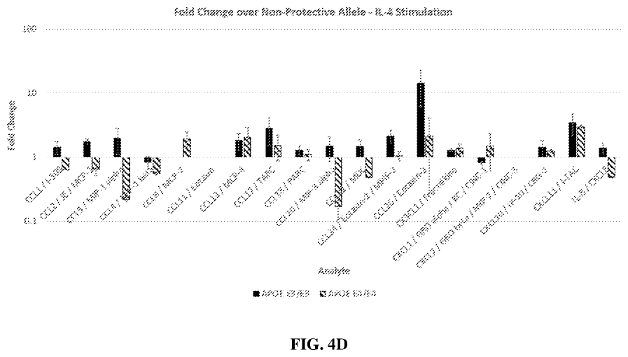

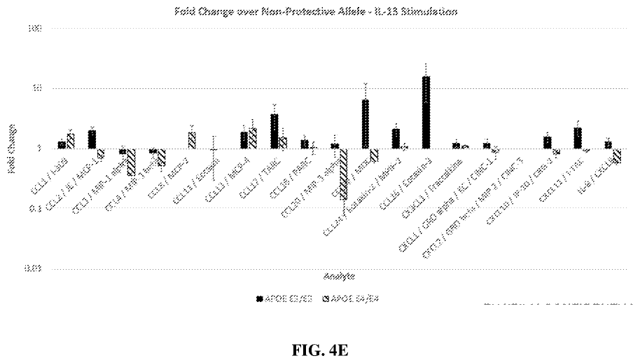

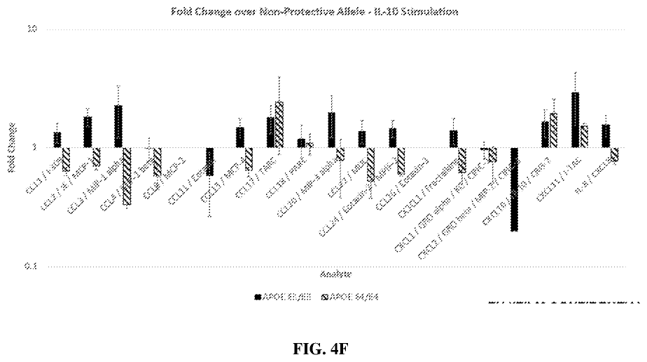

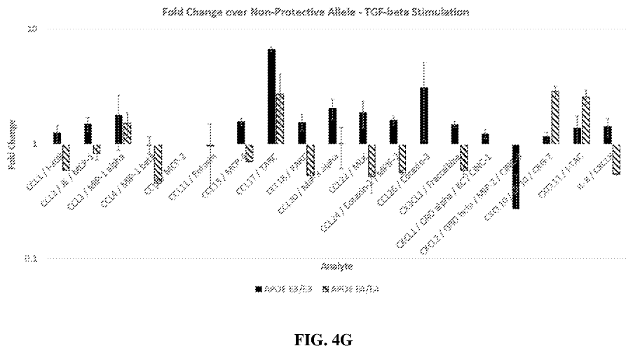

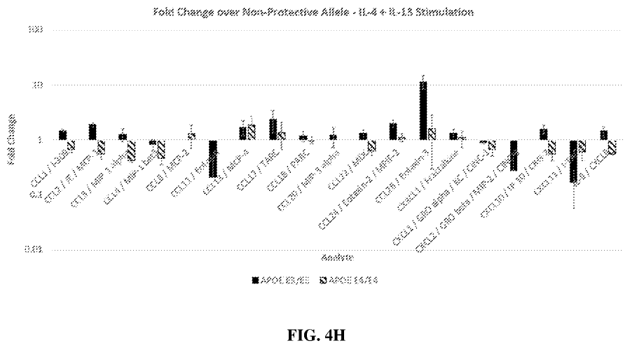

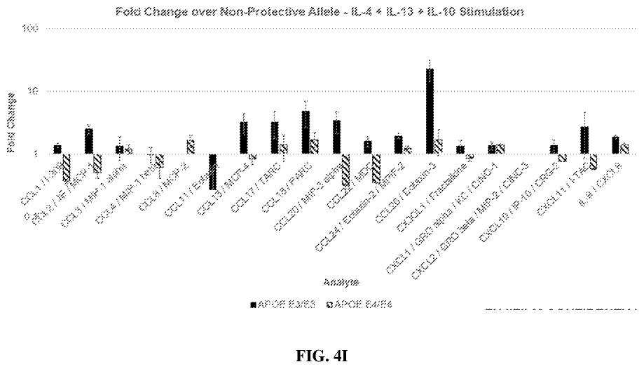

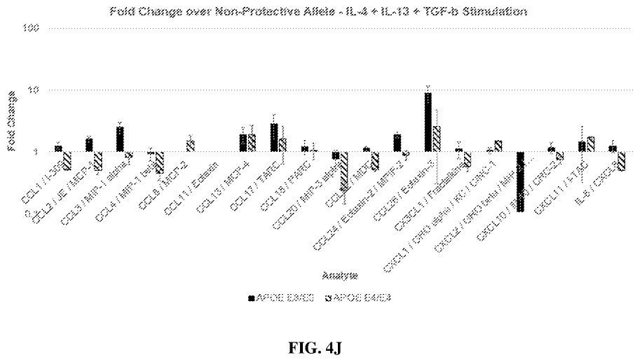

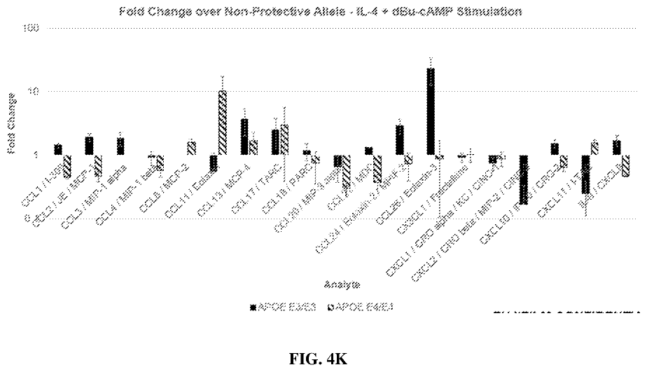

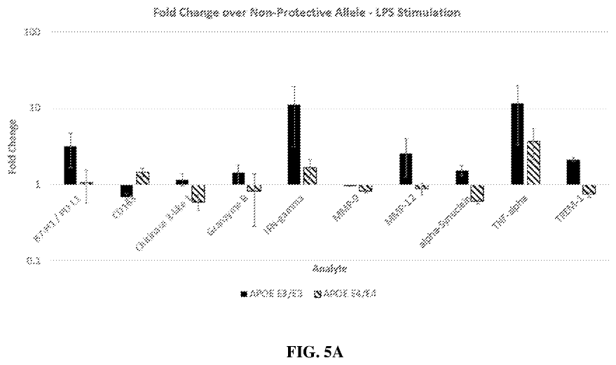

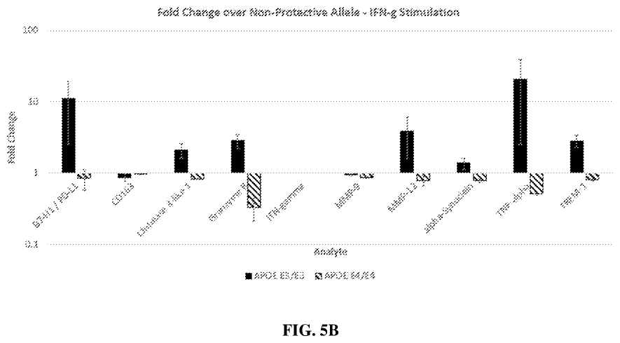

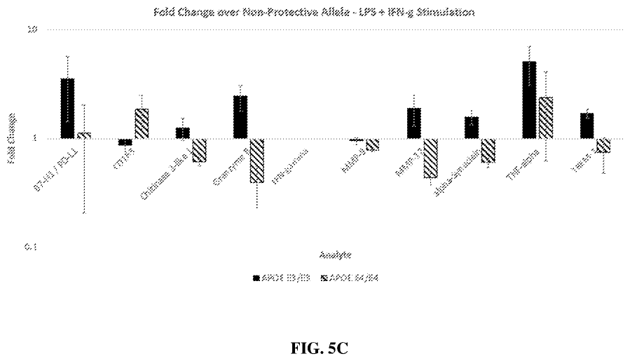

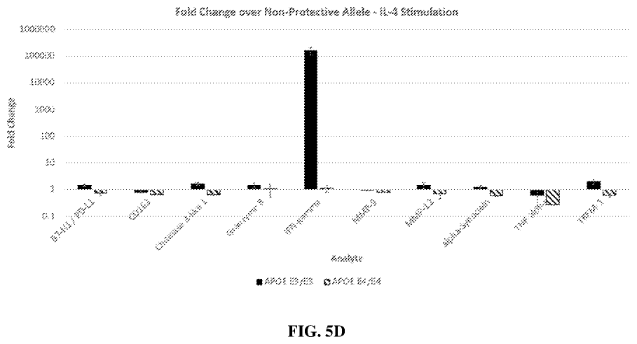

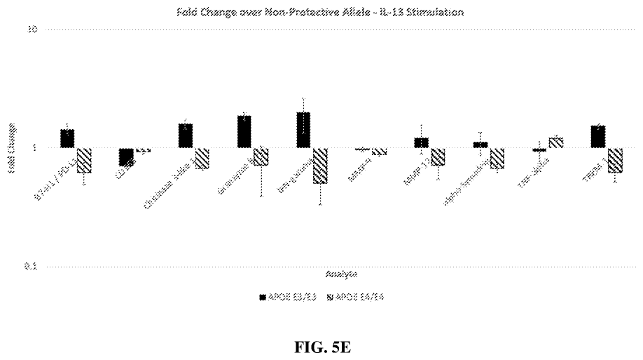

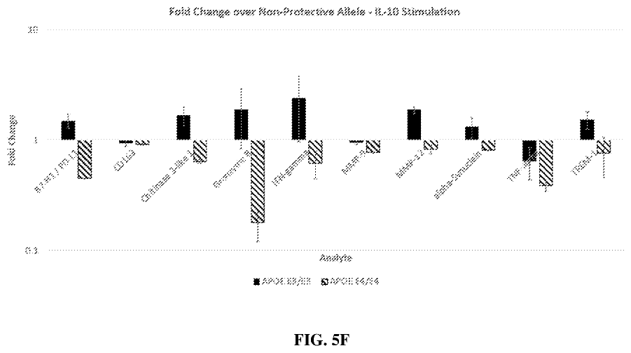

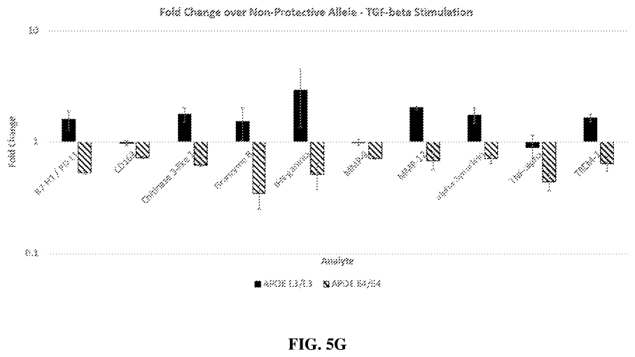

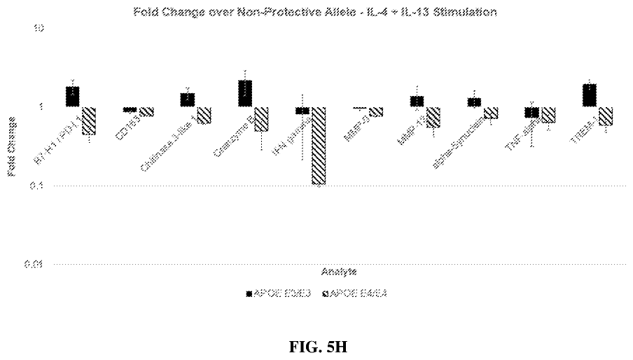

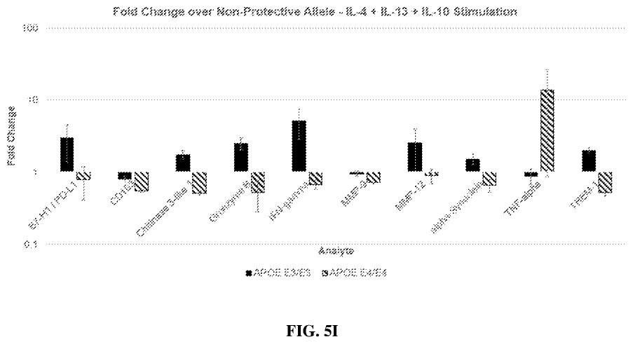

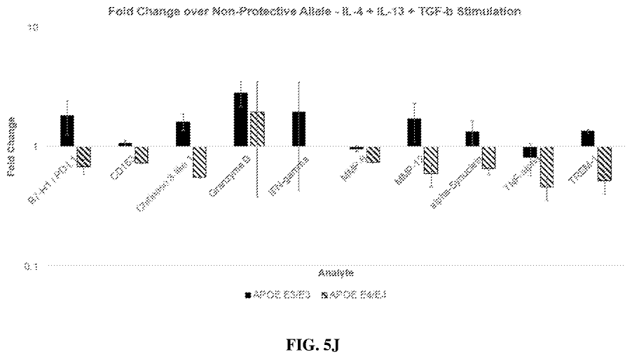

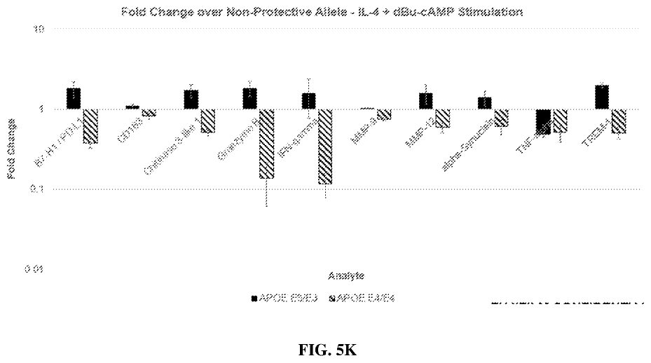

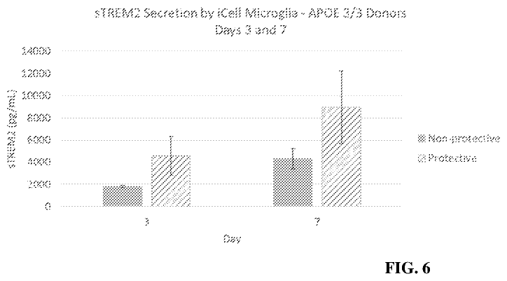

Microglia undergo polarization to M1 or M2 macrophages in response to environmental signals. M1-polarized microglia are activated by the cytokine interferon-γ (IFN-γ), LPS or GM-CSF and produce pro-inflammatory molecules, including tumor necrosis factor (TNF)-α and interleukin (IL)-1, -6, -12, -23 (FIGS. 3-5). M2 polarization stimulated by IL-4, IL-13, or dBu-cAMP is associated with immune regulation, neuroprotection, tissue remodeling and confers protection towards pathogens or tumors. They produce microbicidal and tumoricidal reagents, such as nitric oxide (NO) or reactive oxygen intermediates (ROI).

Cryopreserved iCell microglia were plated in microglia maintenance medium and allowed to recover for 3 days prior to stimulation with LPS to polarize microglia towards the M1 phenotype or with IL+4+dBu-cAMP to polarize microglia towards M2 phenotype for 24 hours. Supernatants were assayed using the multiplex Luminex system in technical duplicates. For each set of analytes, the fold change over unstimulated control was calculated for each cell line, followed by comparison of lines with the protective allele to non-protective lines within either a APOE3/3 or APOE4/4 cohort. Each graph represents the average ±1 SEM. Microglia expressing the protective allele rs12459419 T revealed fold increase in secretion of interleukins IL-27 and IL-10, chemokines CXCL10, CXCL11, CCL1, CCL17, CCL20, and CCL22, and PD-1 ligand expression in AD iPSCs with an APOE 4/4 background.

### Example 2—Pathways and Mechanisms Contributing to the Protective Effect of CD33

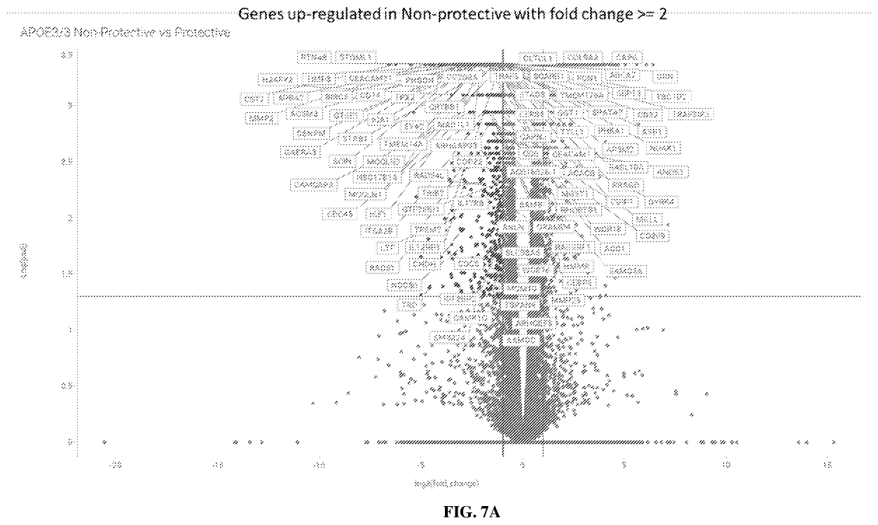

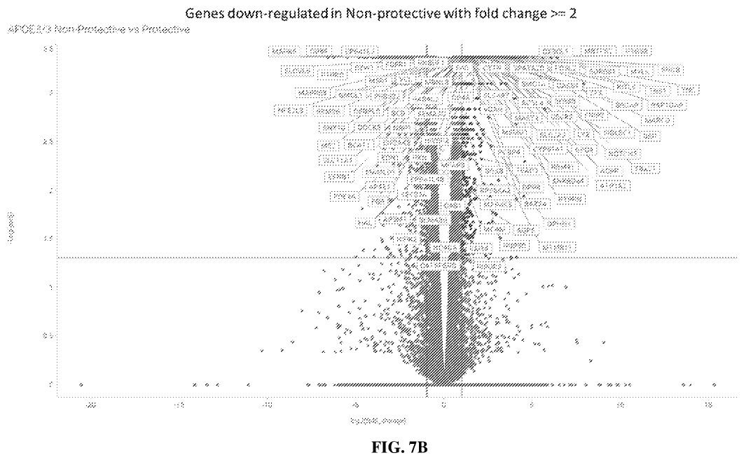

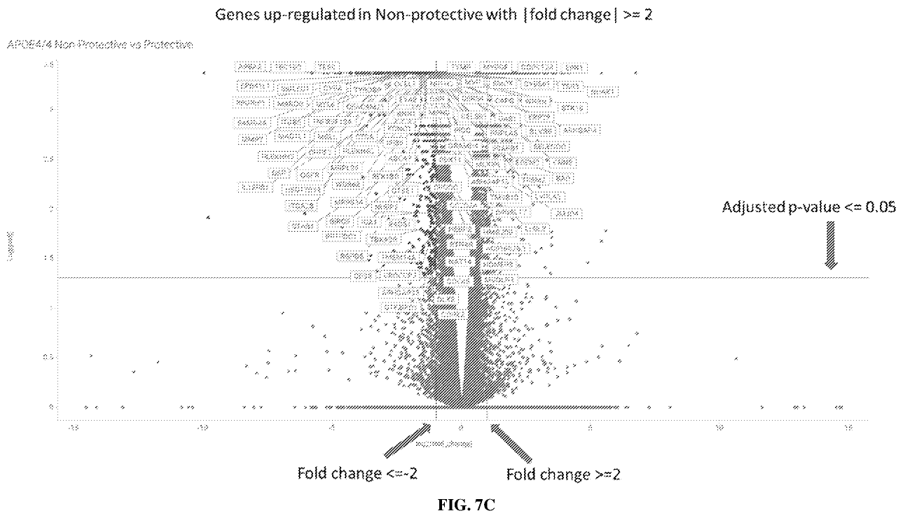

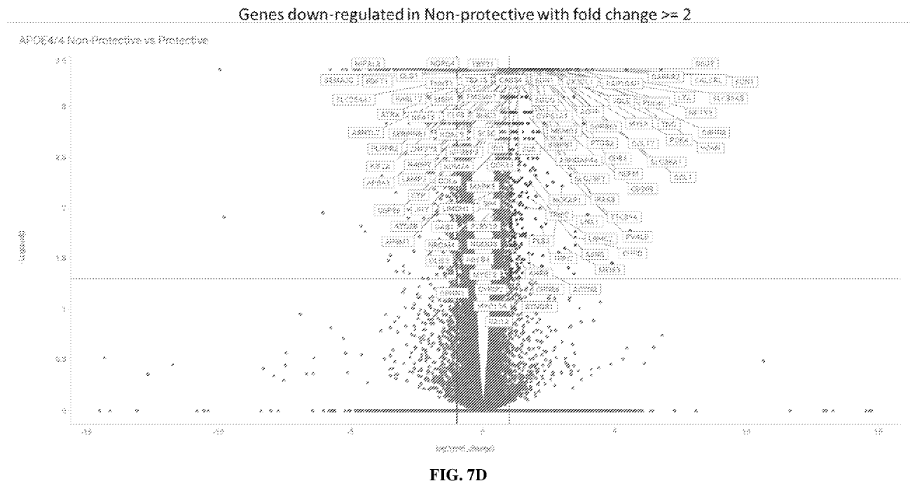

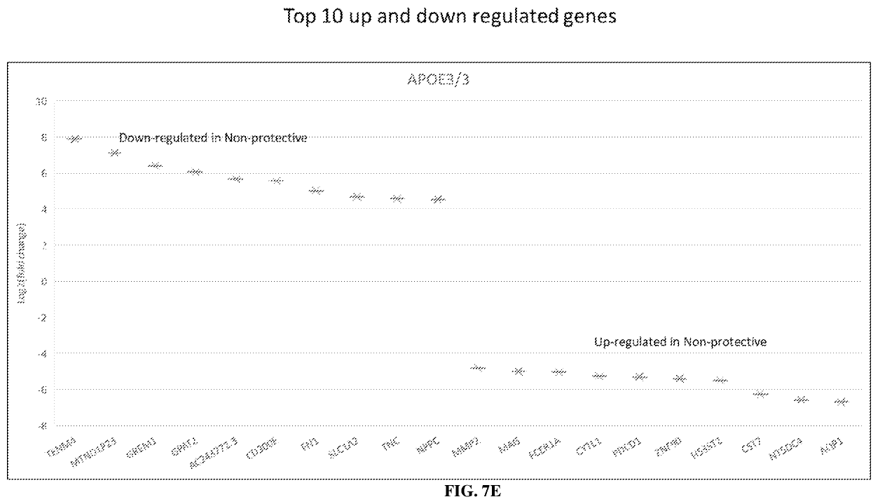

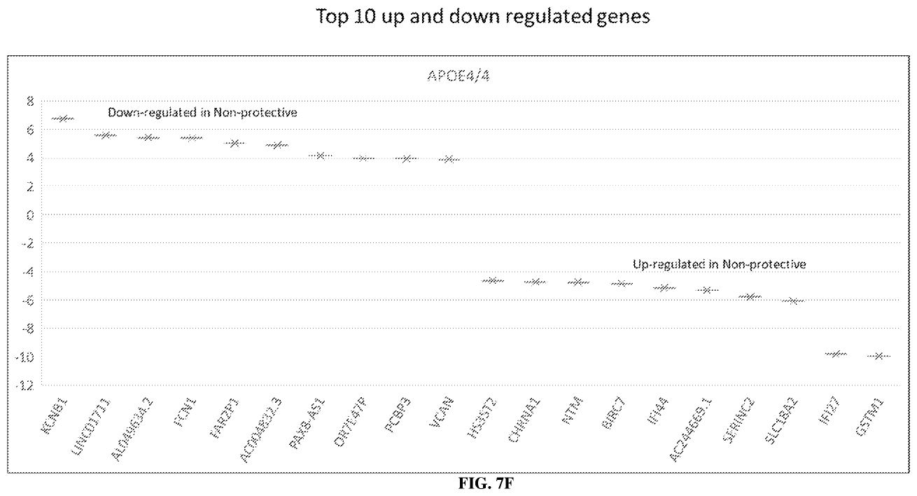

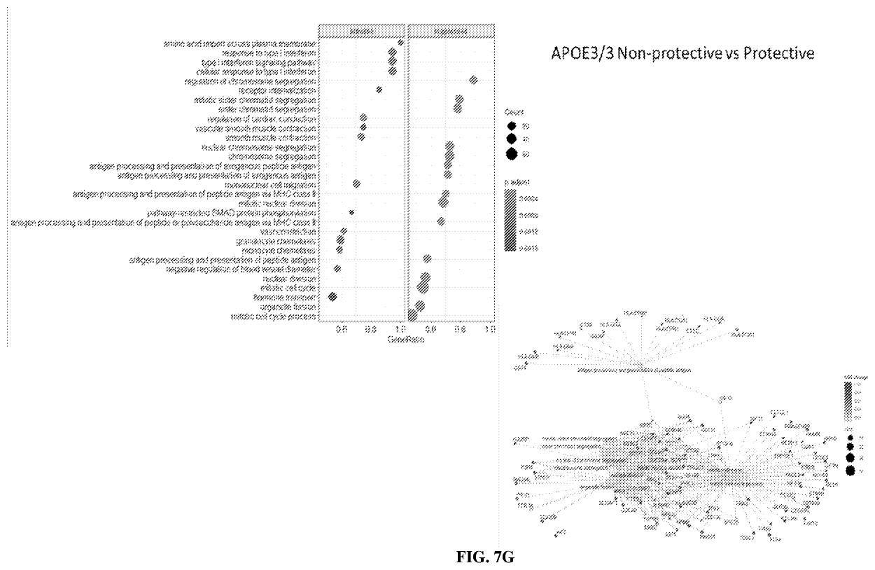

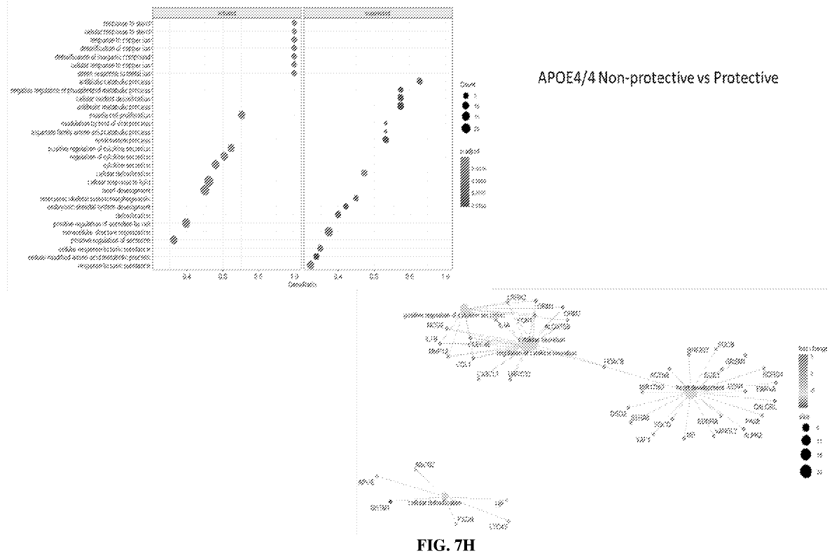

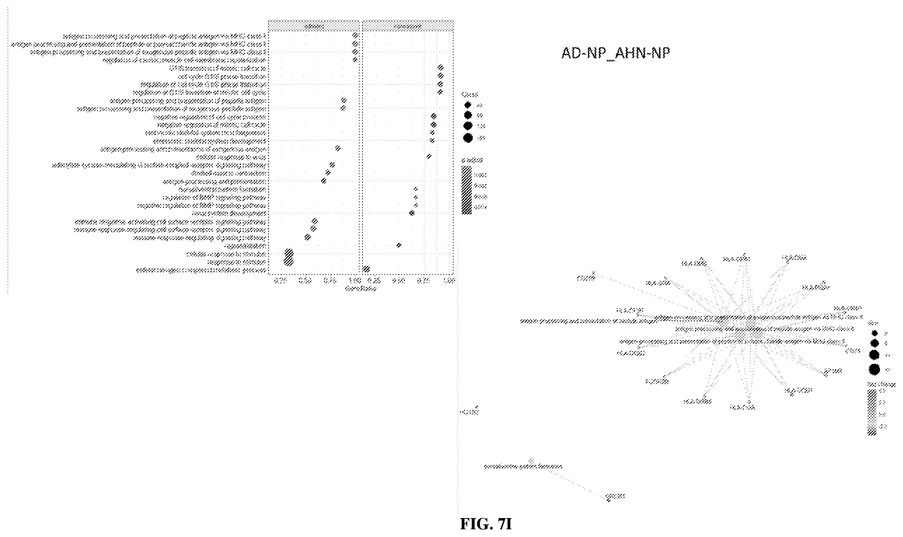

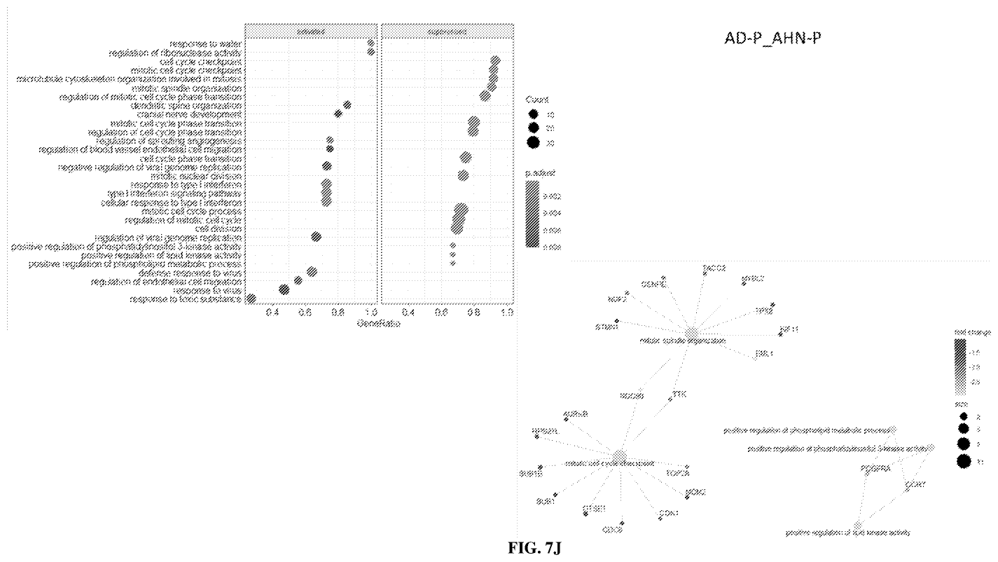

Cryopreserved iCell microglia were plated in microglia maintenance medium and allowed to recover for 3 days and submitted for RNA Seq analysis. The transcriptomic profiles of the iPSC derived microglia with CD33 protective allele over non-protective allele were compared under both APOE3/3 and APOE4/4 backgrounds. The volcano plots of FIGS. 7A-7D show the statistically significant differentially expressed genes at corrected P≤0.05 and |fold change|≥2 highlighted in blue circles (Table 1). FIGS. 7E and 7F show the fold change of the top 10 genes upregulated and downregulated over the non-protective allele in APOE3/3 and APOE4/4 backgrounds.

The Gene concept network plots (CNET plots) and dot plots outlined in FIGS. 7G, 7H, 7I and 7J reveal the results of pathway enrichment analysis. The GO (biological process) term enrichment analysis was performed using the statistically significant (corrected p-value ≤0.05 and |Log 2FC|≥2) genes. Dot size indicated k/n ratio (“gene ratio”), where k is the number of genes participating in the current GO biological process (within the selected gene list), and n is the total number of genes annotated as participants of any GO term. Dot color indicates the enrichment test p-value (Fisher's exact test). The data reveals an insight to many novel mechanisms driven by the protective variant of CD33.

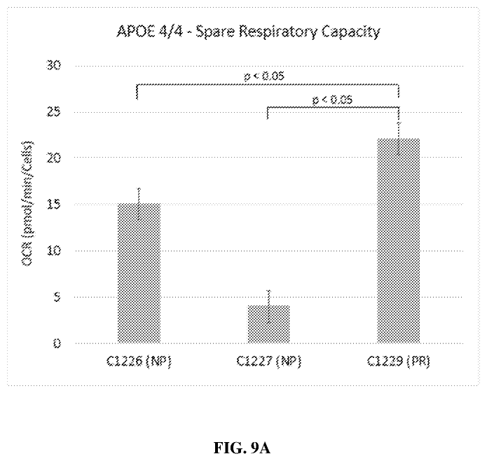

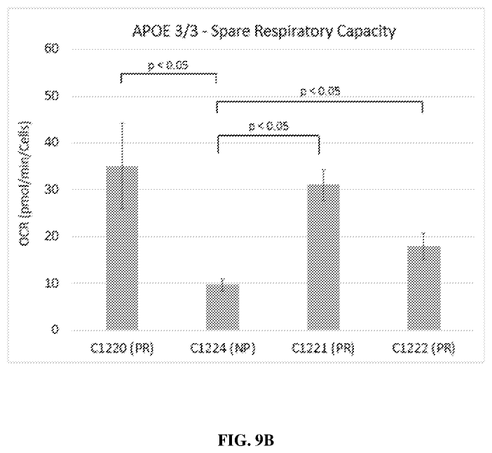

Spare Respiratory Capacity of APOE 3/3 and APOE 4/4 Microglia. Microglia were thawed and rested for three days prior to seeding for an Agilent Seahorse Assay. Microglia were seeded at 20,000 cells per well in a PDL-coated 96-well plate and rested overnight. On the day of assay, medium was exchanged for Assay Medium containing Seahorse XF DMEM, Glucose (10 mM), Sodium Pyruvate (1 mM), and L-Glutamate (2 mM). The plate was then incubated in a 37° C. incubator with ambient C02 for one hour. Stock compounds of Oligomycin A (10 μM), FCCP (30 μM), and Rotenon/Antimycin A (5 μM) from an Agilent Cell Mito Stress Test Kit were prepared and loaded into the appropriate ports of a XF96 Sensor Cartridge, according to Manufacturer's Instructions. Samples were analyzed on an Agilent Seahorse Analyzer with Wave Controller software package. Cell number was determined post-assay using Hoechst nuclear dye (1:1000) and captured using an ImageXpress MetaXpress High Content Imager. Data was normalized to oxygen consumption rate (OCR) per cell. Statistical significance was determined by two-tailed t-test for p<0.05. (FIG. 9A) APOE 3/3 and (FIG. 9B) APOE 4/4 microglia comprising the protective (PR) CD33 (rs12459419) SNP revealed statically significant OCR over microglia comprising the non-protective (NP) CD33 (rs12459419) SNP.

Impairment of mitochondrial metabolism in AD patients has been suggested as a cellular mechanism for the onset and further development of disease phenotype (Bell et al., 2020). In addition, compared to other cell types, microglia have a low mitochondrial turnover, causing impairment of mitochondrial functions to severely affect cell quality and activity (Fairley et al., 2021). Lack of microglial responsiveness, initiated by metabolic deficiency, allows for build up of soluble and oligomeric A-beta in the early stages of Alzheimer's Disease progression, creating a neurotoxic environment (Shippy et al., 2020). It has been shown that the protective allele rs12459419 T and other known protective alleles of CD33 are able to prevent the onset of AD in an APOE 4/4 positive donor. In support, microglia comprising the protective CD33 (rs12459419) SNP revealed a higher oxygen consumption rate than microglia comprising the non-protective CD33 (rs12459419) SNP.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Atagi et al., J Biol Chem. 290(43):26043-50, 2015.
Bell et al., J Pers Med. 10(2):32, 2020.
Brennand et al., 2011
Caldeira et al., Front Aging Neurosci. 9:277, 2017.
Cheng et al., Clin Chim Acta. 463:88-95, 2016.
Ebud et al., Neuron, 94(2):278-293, 2017.
Fairley et al., Front. Immunol., 2021.
International Patent Publication No. WO 02/016536
International Patent Publication No. WO 03/016496
International Patent Publication No. WO 98/30679
International Patent Publication No. WO 98/53058
International Patent Publication No. WO 98/53059
International Patent Publication No. WO 98/53060
Julia et al. Stem cell reports. 9(2): 600-614, 2017.
McQuade et al., J Mol Biol. 431(9):1805-17, 2019.
Melief et al., Glia. 60(10):1506-17, 2012.
Minett et al., J Neuroinflammation. 13(1):135, 2016.
PCT Publication No. 2012/149484
Rustenhoven et al., Trends In Pharmacological Sciences, 38(3), 291-304, 2017.
Shippy et al., Front Cell Neurosci. 14:563446, 2020.
Slosarek et al. Cell Rep. 24(9):2248-2260, 2018.
U.S. patent application Ser. No. 12/715,136
U.S. Pat. No. 6,140,081
U.S. Pat. No. 6,453,242
U.S. Pat. No. 6,534,261
U.S. Pat. No. 6,617,152
U.S. Pat. No. 8,372,642
U.S. Patent Publication No. 2002/0076747
U.S. Patent Publication No. 2005/0064474
U.S. Patent Publication No. 2005/0260186
U.S. Patent Publication No. 2006/0104968
U.S. Patent Publication No. 2006/0188987
U.S. Patent Publication No. 2007/0218528
U.S. Patent Publication No. 2011/0301073
U.S. Patent Publication No. 2011/0301073
U.S. Patent Publication No. 2011/0301073.
Westin et al. PLoS ONE. 7:e30525, 2012.
Winkler et al., Brain pathology (Zurich, Switzerland), 24(4), 371-386, 2014.
Wolfe et al., Int J Mol Sci. 20(1), 2018.

What is claimed is:

1. An isolated induced pluripotent stem cell (iPSC)-derived microglia cell line comprising a CD33 rs12459419T allele or CD33 rs12459419C allele.

2. The cell line of claim 1, wherein the cell line has an APOE 3/3 genotype.

3. The cell line of claim 1, wherein the cell line has an APOE 4/4 genotype.

4. The cell line of any of claims 1-3, wherein the iPSC of the iPSC-derived microglia cell line is an iPSC episomally reprogrammed from a healthy donor.

5. The cell line of any of claims 1-3, wherein the iPSC of the iPSC-derived microglia is an episomally reprogrammed from a donor with Alzheimer's disease.

6. The cell line of any of claims 1-5, wherein cell line expresses CD45, CD11c, CD33, CD11b, and/or TREM2.

7. The cell line of any of claims 1-6, wherein the cell line expresses PU.1, IBA-1, TREM2, CX3CR1, P2RY12, and/or TMEM119.

8. The cell line of any of claims 1-7, wherein the cell line is isogenic.

9. A kit comprising the cell line of any of claims 1-8 in a suitable container.

10. The kit of claim 9, wherein the kit comprises an iPSC-derived microglia cell line comprising a CD33 rs12459419T allele in a first container and an iPSC-derived microglia cell line comprising a CD33 rs12459419C allele in a second container.

11. The kit of claim 10, wherein the cell line has an APOE 3/3 genotype.

12. The kit of claim 10, wherein the cell line has an APOE 4/4 genotype.

13. The kit of claim 9 or 10, further comprising IFNγ, LPS, and/or GM-CSF each in a suitable container.

14. The kit of any of claims 9-13, further comprising IL-4, IL-13, and/or dibutyl cAMP each in a suitable container.

15. The kit of any of claims 9-14, further comprising reagents for detecting the level of IL-27, IL-10, CXCL10, CXCL11, CCL1, CCL17, CCL20, CCL22, and/or PD-1 each in a suitable container.

16. The kit of claim 15, wherein the reagents are further defined as an enzyme-linked immunosorbent assay (ELISA) reagents.

17. The kit of claim 16, further comprising an ELISA plate.

18. A method for screening for a neurodegenerative disease comprising contacting an iPSC-derived microglia cell line comprising a CD33 rs12459419T allele with a sample.

19. The method of claim 18, wherein the cell line has an APOE 3/3 genotype.

20. The method of claim 18, wherein the cell line has an APOE 4/4 genotype.

21. The method of claim 18, further comprising contacting an iPSC-derived microglia cell line comprising a CD33 rs12459419C allele with said sample.

22. The method of claim 18 or claim 21, wherein the iPSC-derived microglia cell line comprising the CD33 rs12459419T allele and/or the iPSC-derived microglia cell line comprising a CD33 rs12459419C allele is a cell line according to any of claims 1-8.

23. The method of any of claims 18-22, wherein the sample is a patient sample.

24. The method of any of claims 18-23, wherein the sample is a blood sample.

25. The method of any of claims 22-24, further comprising detecting the level of IL-27, IL-10, CXCL10, CXCL11, CCL1, CCL17, CCL20, CCL22, and/or PD-1.

26. The method of claim 25, wherein levels of IL-27, IL-10, CXCL10, CXCL11, CCL1, CCL17, CCL20, CCL22, and/or PD-1 indicate the presence or absence of a neurodegenerative disease.

27. The method of any of claims 18-26, wherein the neurodegenerative disease is Alzheimer's disease, Parkinson's disease, Huntington's disease, or multiple sclerosis.

28. A method for screening a test compound comprising introducing the test compound to a microglia cell line of any of claims 1-8 and measuring levels of analytes.

29. The method of claim 28, further comprising measuring amyloid beta phagocytic function.

30. The method of claim 28 or 29, wherein the microglia cell population is further introduced to a pro-inflammatory (M1) agent or an anti-inflammatory (M2) agent.

31. The method of 26, wherein the pro-inflammatory (M1) agent is LPS, IFNγ, and/or GM-CSF.

32. The method of claim 30, wherein the anti-inflammatory (M2) agent is IL-4, IL-13, IL-10 and/or dibutyl cAMP.

33. The method of any of claims 28-32, wherein the analytes are selected from the group consisting of IL-27, IL-10, CXCL10, CXCL11, CCL1, CCL17, CCL20, CCL22, and PD-1.

34. The method of any of claims 28-32, wherein the analytes are IL-27, IL-10, CXCL10, CXCL11, CCL1, CCL17, CCL20, CCL22, and/or PD-1.

35. The method of claim 33, wherein an agent that increases the level of IL-27, IL-10, CXCL10, CXCL11, CCL1, CCL17, CCL20, CCL22, and/or PD-1 is an anti-beta amyloid agent.

36. The method of claim 35, further comprising administering the anti-beta amyloid agent to a subject in an amount effective to prevent or decrease amyloid accumulation.

37. The method of claim 36, wherein the subject is APOE 4/4 positive.

38. A method of identifying a subject at risk for neurodegeneration comprising determining an expression level of at least 10 genes from Table 1A and at least 10 genes Table 1B in a blood sample, wherein a subject with decreased expression of genes in Table 1A and increased expression of genes in Table 1B as compared to a control is at risk for neurodegeneration.

39. The method of claim 38, wherein the at least 10 genes in Table 1A are TENM4, MTND1P23, GREM1, GPAT2, AC243772.3, CD300E, FN1, SLC1A1, TNC, and/or NPPC.

40. The method of claim 38, wherein the at least 10 genes in Table 1B are MMP2, MAG, FCER1A, CYTL1, PDCD1, ZNF90, HS3ST2, CST7, NT5DC4, and/or AQP1.

41. The method of any of claims 38-40, wherein the neurodegeneration is associated with Alzheimer's disease, Parkinson's disease, Huntington's disease, or multiple sclerosis.

42. The method of any of claims 38-41, wherein determining the expression level comprises performing reverse transcription-quantitative real-time PCR (RT-qPCR), microarray analysis, Nanostring® nCounter assay, picodroplet targeting and reverse transcription, or RNA sequencing.

43. The method of any of claims 38-42, further comprising administering an effective amount of a therapy to said subject identified to be a risk for neurodegeneration.

44. The method of claim 43, wherein the therapy is a cholinesterase inhibitor or anti-inflammatory agent.

45. A method for performing high-throughput screening to identify a therapeutic agent comprising contacting a cell line of any of claims 1-9 with a plurality of candidate agents and measuring levels of analytes.

46. The method of claim 45, wherein the analytes are IL-27, IL-10, CXCL10, CXCL11, CCL1, CCL17, CCL20, CCL22, and/or PD-1.

47. The method of claim 46, further comprising measuring amyloid beta phagocytic function.

48. A co-culture comprising a microglia cell line of any of claims 1-8, and endothelial cells, pericytes, astrocytes, and/or neural precursor cells.

49. Use of the co-culture of claim 48 as a model of a neurodegenerative disease.

50. A composition comprising a microglia cell population at least 90% positive for TREM2, CD45, CD11c, CD33, CD11b, PU.1, IBA-1, TREM2, CX3CR1, P2RY12, and/or TMEM119, wherein the microglia cell population is differentiated from iPSCs comprising a CD33 rs12459419T allele or CD33 rs12459419C allele.

51. The composition of claim 50, wherein the microglia cell population is differentiated from iPSCs comprising a CD33 rs12459419T allele.

52. The composition of claim 50, wherein the microglia cell population is differentiated from iPSCs comprising a CD33 rs12459419C allele.

53. The composition of any of claims 50-52, wherein the microglia cell population has an APOE 3/3 genotype.

54. The composition of any of claims 50-53, wherein the microglia cell population has an APOE 4/4 genotype.

55. Use of the composition of any of claims 50-54 for identifying a subject at risk for neurodegeneration.

56. The use of claim 55, wherein identifying a subject at risk comprises determining an expression level of at least 10 genes from Table 1A and at least 10 genes Table 1B in a blood sample, wherein a subject with decreased expression of genes in Table 1A and increased expression of genes in Table 1B as compared to a control is at risk for neurodegeneration.

57. The use of claim 56, wherein the at least 10 genes in Table 1A are TENM4, MTND1P23, GREM1, GPAT2, AC243772.3, CD300E, FN1, SLC1A1, TNC, and/or NPPC.

58. The use of claim 56, wherein the at least 10 genes in Table 1B are MMP2, MAG, FCER1A, CYTL1, PDCD1, ZNF90, HS3ST2, CST7, NT5DC4, and/or AQP1.

59. The use of any of claims 55-58, wherein the neurodegeneration is associated with Alzheimer's disease, Parkinson's disease, Huntington's disease, or multiple sclerosis.

60. The use of any of claims 56-59, wherein determining the expression level comprises performing reverse transcription-quantitative real-time PCR (RT-qPCR), microarray analysis, Nanostring® nCounter assay, picodroplet targeting and reverse transcription, or RNA sequencing.

61. The use of any of claims 55-60, further comprising administering an effective amount of a therapy to said subject identified to be a risk for neurodegeneration.

62. The use of claim 61, wherein the therapy is a cholinesterase inhibitor or anti-inflammatory agent.

63. Use of the composition of any of claims 50-54 for screening a test compound comprising introducing the test compound to the composition and measuring levels of analytes.

64. The use of claim 63, further comprising measuring amyloid beta phagocytic function.

65. The use of claim 63 or 64, wherein the microglia cell population is further introduced to a pro-inflammatory (M1) agent or an anti-inflammatory (M2) agent.

66. The use of 63, wherein the pro-inflammatory (M1) agent is LPS, IFNγ, and/or GM-CSF.

67. The use of claim 65, wherein the anti-inflammatory (M2) agent is IL-4, IL-13, IL-10 and/or dibutyl cAMP.

68. The use of any of claims 63-67, wherein the analytes are selected from the group consisting of IL-27, IL-10, CXCL10, CXCL11, CCL1, CCL17, CCL20, CCL22, and PD-1.

69. The use of any of claims 63-67, wherein the analytes are IL-27, IL-10, CXCL10, CXCL11, CCL1, CCL17, CCL20, CCL22, and/or PD-1.

70. The use of claim 68, wherein an agent that increases the level of IL-27, IL-10, CXCL10, CXCL11, CCL1, CCL17, CCL20, CCL22, and/or PD-1 is an anti-beta amyloid agent.

71. The use of claim 70, further comprising administering the anti-beta amyloid agent to a subject in an amount effective to prevent or decrease amyloid accumulation.

72. The use of claim 71, wherein the subject is APOE 4/4 positive.
